(19) United States
(12) Patent Application Publication
(10) Pub. No.: US 2024/0051999 A1
(43) Pub. Date: Feb. 15, 2024

(54) METHODS FOR TARGETED PROTEIN DEGRADATION

(71) Applicants: JOHN INNES CENTRE, Norwich (GB); THE SAINSBURY LABORATORY, Norwich (GB)

(72) Inventors: Saskia Hogenhout, Norwich (GB); Weijie Huang, Norwich (GB); Allyson MacLean, Norwich (GB); Akiko Sugio, Norwich (GB); Sophien Kamoun, Norwich (GB); Abbas Maqbool, Norwich (GB)

(21) Appl. No.: 18/267,631

(22) PCT Filed: Dec. 17, 2021
(86) PCT No.: PCT/EP2021/086666

§ 371 Date: Jun. 15, 2023

(30) Foreign Application Priority Data
Dec. 18, 2020 (LU) 102306
Aug. 25, 2021 (GB) 2112172.8

(51) Int. Cl. C07K 14/195; C12N 15/11; C12N 9/22; C12N 15/90; C12N 15/82

(52) U.S. Cl. CPC C07K 14/195; C12N 15/11; C12N 9/22; C12N 15/907; C12N 15/8213; C12N 15/8251; C12N 2310/20; C12N 2800/107

(57) ABSTRACT

The present invention relates to a fusion protein for use in ubiquitin-independent protein degradation. The invention also relates to methods for the use of the fusion protein in targeted protein degradation, modulating physiological responses and therapy.

### FIELD OF THE INVENTION

The present invention relates to a fusion protein for use in ubiquitin-independent protein degradation. The invention also relates to methods for the use of the fusion protein in targeted protein degradation, modulating physiological responses and therapy.

### BACKGROUND OF THE INVENTION

It is possible to reduce protein levels in a targeted manner by disruption at the transcription level through nucleic acid-based tools such as RNA interference (RNAi) and more recently, CRISPR/Cas9 gene editing technology. However, these technologies have specific disadvantages, such as (embryonic) lethality, irreversibility, lack of tissue specificity and off-target effects. In addition, safe and efficient cell delivery is an issue, as well as metabolic stability and problematic off-target effects.

In view of these issues, targeted protein degradation (TPD) has been explored to reduce protein in cells, which work at the post-translational level to cause degradation. An advantage of this approach is that target proteins can be accurately and rapidly degraded in a tissue/cell specific manner, even if the target protein has a slow turnover rate. This is favourable over degradation at the transcriptional level as experiments using these tools can be difficult to interpret due to the delay between loss of gene expression and resulting protein depletion. Specifically targeting at the protein level also avoids adaption events at the genomic level, such as gene amplification and also offers isoform specificity.

Targeting at the protein level can also be very advantageous where a target has a function in more than one stage of development of the organism. By targeting degradation at the cell or tissue level it is possible to selectively degrade proteins in many useful contexts. For example, in plants there are a number of transcription factors that have functions in both shoot/root meristem and embryo development and it is difficult to study the function of these proteins in meristems if the plant dies at the embryo stage. Therefore, by specifically targeting proteins at a tissue level, it would be possible to deplete the protein only in the meristem, and not in the embryo. In a similar way, Toll-like receptors are important during embryo development in Drosophila but also have a key role in immunity, therefore conducting protein degradation after embryo development allows for the investigation of these proteins specifically in the context of immunity without causing harmful effects on development.

Clearly, there are numerous applications for a selective protein degradation technology in both plants and animals, as well as in yeasts and fungi, where it is desirable and/or useful to reduce or eliminate the presence of a particular protein of interest.

In animal systems, recent examples include Specific and Nongenetic Inhibitor of Apoptosis Protein (IAP)-dependent Protein Erasers (SNIPERs), proteolysis-targeting chimeras (PROTACs), and Degronimids, which have offered the ability to target previously “undruggable” targets that were not capable of pharmacological targeting. Of note, all of these examples induce selective degradation of target proteins through ubiquitin-mediated pathways. Ubiquitination is an enzymatic process involving three steps: activation, conjugation, and ligation; and requiring three enzymes: ubiquitin-activating enzymes (E1s), ubiquitin-conjugated enzymes (E2) and ubiquitin ligases (E3s) (6). Firstly, ubiquitin, a small (8.6 kA) regulatory protein is activated by E1s through an ATP dependent reaction. Secondly, the transfer of ubiquitin from E1 to E2 occurs through catalyzation by E2. Lastly, E3s catalyse the production of an isopeptide bond between a lysine of the target protein and the C-terminal glycine of ubiquitin. Further ubiquitin molecules can then be added to the first, creating a polyubiquitin chain which marks the protein for targeted protein destruction by the proteasome. Ubiquitin mediated degradation therefore requires this energy intensive three step enzymatic process to be repeated many times over to effectively degrade the protein. Harnessing this process for targeted protein degradation has also been technically challenging, due to the highly regulated nature of ubiquitination, which depends on cell or tissue specific enzymes. These enzymes need to be identified and characterized in the tissues/cells, and it can be highly challenging to identify the right combination of enzymes to be effective for a given substrate. Even when these enzymes are identified, it is difficult to exclude that these enzymes are active elsewhere in an organism, which increases the risk of off-target effects. It is also possible that ubiquitin chains can be of different compositions, with some ubiquitin compositions not being degraded but instead being transported to another cell location, such as the nucleus.

There is therefore a need to develop alternative selective protein degradation technologies that bypasses the need for ubiquitination. The present invention addresses this need.

### SUMMARY OF THE INVENTION

We have developed a protein knockdown technology that can induce selective proteasomal protein degradation independent of ubiquitin. Specifically, we have identified an effector that directly binds a 26S proteasome component leading to the degradation of substrates, without the requirement for ubiquitination. To date, effectors that directly link host targets to the proteasome system in a ubiquitin independent manner are not known. Thus, the mode of action we have identified involving Von Willebrand factor type A domain (vWA)-binding proteins, such as SAP05, is significant. As discussed above, whereas cellular protein levels may be altered by gene knockout and RNA silencing, some systems require the direct targeting of proteins for degradation. In fact, targeted protein degradation has become one of the most promising approaches for drug discovery in targeted therapies. Current approaches to changing protein abundance in cells rely on substrate ubiquitination: for example, the proteolysis-targeting chimera (PROTAC) technique uses small-molecule ligands that create complexes between E3 ligases and targets, a process that can be challenging. Our study of phytoplasma effectors has revealed an alternative approach whereby bridging targets directly onto proteasome subunits, such as RPN10—and specifically the vWA domain of RPN10, results in efficient protein degradation. Through further analyses we have identified that the region of vWA that is on the opposite site of the area of vWA that binds the 26S proteasome is responsible for binding SAP05. Specific mutations of two amino acids within this SAP05-binding region of vWA prevents binding of SAP05 to vWA, but do not prevent binding of vWA to the proteasome. On this basis, we have established that any agent that binds the region of vWA that is on the opposite site of the area of vWA that binds the 26S proteasome can be used to mediate degradation of the target protein, through the proteasome, but independently of ubiquitin.

In one aspect of the invention there is provided a fusion protein comprising a vWA targeting moiety and a target-binding moiety. A vWA targeting moiety can also be called a vWA binding moiety, and such terms are interchangeable. In one embodiment, the vWA targeting moiety binds at least the region that includes residues 38 and 39 of SEQ ID NO: 21, 24 or 26 or corresponding regions and positions in a homologous sequence. In a further embodiment, the vWA targeting moiety binds at least the region of vWA that is on the opposite site of the area of vWA that binds the 26S proteasome. In a further embodiment, the vWA targeting moiety binds only the GA or HS residues or the corresponding residues in a homologous sequence.

In one embodiment, the vWA targeting moiety is a SAP05 peptide. In one embodiment, the SAP05 peptide comprises an amino acid sequence as defined in SEQ ID NO: 1 or a functional variant thereof.

In another embodiment, the fusion protein further comprises an RPN10 peptide, preferably as defined in SEQ ID NO: 5, 24 or 26 or a functional variant thereof and more preferably a vWA domain, preferably as defined in SEQ ID NO: 21 or a functional variant thereof.

In one embodiment, the target-binding moiety is selected from an antibody or antigen-binding fragment thereof or an aptamer. Similarly, in another embodiment, the vWA binding moiety is selected from an antibody or antigen-binding fragment thereof or an aptamer. In one example, the fusion protein may be a bispecific antibody, where one arm of the bispecific antibody binds vWA and the other arm binds the target protein. In an alternative embodiment, the fusion protein may comprise a first antibody that can bind the vWA domain and a second antibody that can bind a target protein, where the first and second antibodies are linked, for example with a linker.

In one embodiment, where the vWA binding moiety is SAP05, the fusion protein further comprises a linker, linking the SAP05 peptide and target-binding moiety

In another aspect, there is provided an expression vector comprising a nucleic acid encoding the fusion peptide of the invention, or a nucleic acid comprising the nucleic acid sequence as defined in SEQ ID NO: 2 and/or 22 or a functional variant thereof.

In a further embodiment, the expression vector further comprises a nucleic acid encoding a plant RPN10 protein, preferably wherein the nucleic acid comprises the nucleic acid sequence as defined in SEQ ID NO: 6 or a functional variant or homologue thereof. In an alternative embodiment, the expression vector further comprises a nucleic acid encoding a vWA domain, preferably wherein the nucleic acid comprises the nucleic acid sequence as defined in SEQ ID NO: 22, 23 or 25 or a functional variant thereof.

In another aspect of the invention, there is provided a cell comprising the fusion protein of the invention or the expression vector of the invention. Preferably the cell is a eukaryotic cell.

In another aspect of the invention, there is provided a transgenic organism expressing the expression vector of the invention or comprising the cell of the invention, wherein the organism is not a human.

In some embodiments, the fusion protein or the expression vector is for use as a medicament. In another embodiment, the fusion protein or expression vector can be used as a biological agrochemical. For example, the fusion protein or expression vector can be used to control plant pests, weeds and other plant pathogens.

In another aspect of the invention, there is provided the use of the fusion protein of the invention or an expression vector of the invention in targeted protein degradation. Alternatively, there is provided the use of a SAP05 protein alone or in combination with a vWA domain in targeted protein degradation. Preferably, the targeted protein degradation is ubiquitin-independent protein degradation.

In another aspect of the invention there is provided a method of targeted protein degradation, the method comprising applying the fusion protein of the invention or the expression vector of the invention to a sample. Alternatively, there is provided a method of protein degradation, the method comprising applying a SAP05 protein alone or in combination with a vWA domain. Preferably, the targeted protein degradation is ubiquitin-independent protein degradation.

In another aspect of the invention, there is provided a method of controlling the level of a target protein, the method comprising applying the fusion protein of the invention, the expression vector of the invention or a SAP05 protein (and optionally additionally a vWA domain) or to a sample.

In one embodiment, a fluorescently tagged antibody is applied to the sample in order to label a protein of interest prior to applying the fusion protein, wherein the target-binding moiety of the fusion protein is specific for the fluorescent tag, and wherein degradation of the labelled protein of interest in the sample can be detected by a decrease in fluorescent signal.

In another embodiment, the target protein is a cytosolic protein or a cell surface protein. In another embodiment, the method may further comprise the step of administering a vWA targeting moiety. The purpose of administering a vWA targeting moiety alone is to prevent prolonged degradation of the target protein by the fusion protein, which could lead to toxicity. Hence, a vWA binding moiety may be added to control or limit the amount of fusion protein-mediated degradation by competing for binding to the region at which the fusion protein binds vWA (or RPN10), thereby preventing degradation. As such, in this embodiment, the vWA targeting binding moiety binds the same region on vWA as the fusion protein. More preferably, the vWA targeting moiety may be added in an amount that is higher than the fusion protein—i.e. in excess—to outcompete binding of the fusion protein to vWA.

In another aspect of the invention, there is provided a method of gene editing, the method comprising:
a) introducing a CRISPR-Cas (or Cpf) system comprising a CRISPR enzyme to a cell or organism,
b) allowing the CRISPR-Cas (or Cpf) system to edit one or more target nucleic acid sequence,
c) delivering a fusion protein or expression vector according to the invention comprising a target moiety that is specific for said CRISPR enzyme; and
d) allowing the fusion protein to degrade the CRISPR enzyme so as to inhibit any further activity of the CRISPR-Cas system.

In one embodiment, the target protein can cause pathology in a target organism or the target protein may be a drug target.

In another aspect of the invention, there is provided a method of modulating a physiological response in an organism, the method comprising administering to said organism a fusion protein of the invention, an expression vector of the invention (encoding the fusion protein) or a SAP05 protein (and optionally additionally a vWA domain). The physiological response may be selected from a stress response, an immune response, a hormone response or a light response. For example, the stress response may be an abiotic or biotic stress response in plants; an example of the latter is a response to a plant pathogen.

In another aspect of the invention there is provided a method of treating a condition in a patient in need thereof, the method comprising administering to said patient a fusion protein of the invention or an expression vector of the invention or a SAP05 protein (and optionally additionally a vWA domain). In one embodiment, the condition is characterised by increased expression or activity of a target protein, and the target-binding moiety is specific for said target protein.

In another aspect of the invention, there is provided a method of treating an infection caused by a microorganism, the method comprising administering to a subject a fusion protein of the invention or an expression vector of the invention, or a SAP05 protein (and optionally additionally a vWA domain), wherein the target binding moiety is specific for a protein expressed by a microorganism.

In another aspect of the invention there is provided a method of increasing the immunogenicity of a protein, the method comprising administering to a subject a fusion protein of the invention or an expression vector of the invention, or a SAP05 protein (and optionally additionally a vWA domain), wherein the target binding moiety is specific for the protein, and wherein proteasome degradation of the protein results in increased antigen presentation of peptides degraded from the protein. In one embodiment, the fusion protein is administered to a subject suffering from an infection or cancer.

In another aspect, there is provided a method for creating a protein knockout model, the method comprising administering a fusion protein of the invention or an expression vector of the invention, or a SAP05 protein (and optionally additionally a vWA domain) to a cell or an organism. In one embodiment, the model is a disease model where the disease is caused or characterised by a dysfunction or absence of a target protein, and the target moiety is specific for the target protein.

In another aspect, there is provided a method of identifying a degradation effect of a target protein in a biological system, the method comprising applying a fusion protein of the invention, an expression vector of the invention (encoding the fusion protein) or a SAP05 protein (and optionally additionally a vWA domain) to the biological system in order to degrade said target protein. Preferably, the target protein is a regulatory protein, and the method further comprises performing RNA sequencing of the RNA of the biological system to determine the transcription status of one or more genes, preferably the targeted gene, possibly a downstream member of a pathway involving said targeted gene, and/or to assess the transcriptome of the system. Preferably, this will further comprise performing RNA sequencing of the RNA of a biological system wherein the method comprising applying the fusion protein of the invention, a SAP05 protein (and optionally additionally a vWA domain), or an expression vector of the invention to the biological system has not been performed (i.e. a control biological system) and comparing the two systems. In some embodiments, the method may be applied as part of a biological or pharmaceutical screening method; for example, by identifying the degradation effect in the presence or absence of a candidate drug, and comparing with a control. In some embodiments the screen may be carried out in a cell line, for example a human cell line, or may be carried out in a model organism, for example, a mouse, or may be carried out in a fungus, for example, a yeast.

In another aspect of the invention there is provided a method of modulating a characteristic of a fungus, the method comprising introducing the fusion protein of the invention, an expression vector of the invention (encoding the fusion protein), or a SAP05 protein (and optionally additionally a vWA domain) to the fungus, wherein the target-binding moiety is specific for a target protein the expression of which is associated with the characteristic. In some embodiments, the fungus is a yeast (eg, Saccharomyces, Candida, Pichia, etc). In some embodiments, the fungus is detrimental to food production; for example, can cause spoilage of food, or may cause infections or ill-health in an organism that may consume the food. In some embodiments, the characteristic to be modulated is growth and/or reproduction of the fungus. In other embodiments, the characteristic to be modulated is production and expression of a toxic compound; or is production and expression of a compound which alters the taste of a food product.

In another aspect of the invention there is provided a method of increasing production in a farm animal, the method comprising introducing the fusion protein of the invention, an expression vector of the invention (encoding the fusion protein) or a SAP05 protein (and optionally additionally a vWA domain) to the farm animal, wherein the target-binding moiety is specific for a target protein the expression or activity of which is negatively correlated with production. In some embodiments the farm animal may be a bovid, caprid, ovid, porcid. In other embodiments the farm animal may be avian; for example, a galloanserine such as chicken, duck.

In another aspect of the invention there is provided a method of increasing yield in a plant, the method comprising introducing the fusion protein of the invention, an expression vector of the invention (encoding the fusion protein), or a SAP05 protein (and optionally additionally a vWA domain) to the plant, wherein the target-binding moiety is specific for a target protein the expression or activity of which is negatively correlated with yield.

In another aspect of the invention there is provided a method of reducing or removing glutens in a plant, the method comprising introducing the fusion protein of the invention, an expression vector of the invention (encoding the fusion protein), or a SAP05 protein (and optionally additionally a vWA domain) to the plant, wherein the target-binding moiety is specific for gliadin or glutenin.

In a preferred embodiment, targeted protein degradation is ubiquitin-independent protein degradation.

In another aspect, there is provided a pharmaceutical composition comprising the fusion protein of the invention, an expression vector of the invention (encoding the fusion protein), or a SAP05 protein (and optionally additionally a vWA domain) and a pharmaceutically acceptable diluent, carrier or excipient. In one embodiment, the pharmaceutical composition is for use in treatment of cancer, infection, a neurodegenerative disorder or a proteopathy.

In the above embodiments where a SAP05 and a vWA domain are used in combination, SAP05 and vWA may be applied or administered separately, sequentially or concurrently.

In another aspect, there is provided a screening library comprising a plurality of fusion proteins according to the invention, wherein each fusion protein comprises a different target-binding moiety.

In another aspect, there is provided a method for screening target-binding moieties using the screening library of the invention. Said methods may be useful in the pharmaceutical industry, for example to screen candidate target-binding moieties as potential therapeutics.

In a final aspect, there is provided a kit comprising a fluorescently tagged antibody and a fusion protein of the invention, wherein the target-binding moiety of the fusion protein is specific for the fluorescent tag.

### DESCRIPTION OF THE FIGURES

The invention is further described in the following non-limiting figures:

FIG. 1—SAP05 degrades plant GATA and SPL transcription factors via the plant 26S proteasomal ubiquitin receptor RPN10. a, Locations of the zinc-finger (ZnF) domains in representative proteins of the A. thaliana (At) GATA and SPL transcription factor families. SBP, SQUAMOSA promoter-binding protein. Red and yellow dots indicate cysteine and histidine residues, respectively. b, SAP05 interacts with AtGATAs and SPLs via their ZnF domains in yeast. EV, empty vector control. AD, GAL4-activation domain. BD, GAL4-DNA binding domain. Yeast transformed with AD and BD constructs grew on medium lacking leucine and tryptophan (-L-W) and growth on medium lacking leucine, tryptophan, histidine and alanine (-L-W-H-A) indicates positive interactions. c, Western blot analysis of SAP05-mediated degradation of GATA and SPL proteins in A. thaliana protoplasts. GFP, control; HA, hemagglutinin tag; rSPL, miR156-resistant form. Numbers at left indicate markers in kilodaltons. d, Domain localisation of A. thaliana RPN10. vWA, von Willebrand factor type A domain; UIM, ubiquitin-interacting motifs. e, SAP05 interacts with AtRPN10 via its vWA domain in yeast. N-ter and C-ter refer to the N and C termini, respectively. f-k, Western blot analysis of SAP05 degradation assays in either rpn10-2 (f) or wild type (g-k) A. thaliana protoplasts. SAP05-mediated degradation of targets is dependent on AtRPN10 (f) and independent of the presence of lysines in targets (g). K->R, all lysines replaced by arginines (g). In h-k, ‘tail’ represents an unstructured region that serves as an initiation site for proteasomal degradation. (a,d) Numbers below indicate amino acid positions. (c,f-k) Red dots at left of bands indicate expected protein sizes.

FIG. 2—SAP05 degrades SPL and GATA transcription factors in whole plants. a-d, SAP05 suppresses plant phenotypes caused by the overexpression of various SPL or GATA members; shown are plant phenotypes (a) and quantitative analyses of leaf serrations (b), rosette leaf numbers (c) and shoot lengths (d). OE, overexpression; x, cross of two lines; rSPL, miR156-resistant form. In a, arrowheads indicate leaf serrations, and arrow a flowering stem; scale bars, 1 cm. Each data point (black or red dot) represents one transgenic line, and the columns show means±s.d. of these data points. *, P<0.05, two-tailed unpaired Student's t-tests. e-f, SPLs (SPL11, e, and SPL13, f) are degraded in plants infected with AY-WB phytoplasma. Black arrows indicate blue GUS staining of non-infected plants that is strongly reduced in AY-WB-infected plants. g shows a schematic illustration of SAP05-GFP nanobody fusion protein where the SAP05 coding sequence and the GFP nanobody coding sequencing was fused with a GS linker.

FIG. 3—SAP05 functions via the proteasome and not via autophagy. 26S proteasome inhibitors MG132 and bortezomib suppress SAP05-mediated protein destabilization. (A and B). Western blot analysis of SAP05-mediated GATA18 (A) and SPL5 (B) degradation in A. thaliana protoplasts in the presence of either 26S proteasome inhibitors (MG132 and bortezomib), autophagy inhibitors (3-Methyladenine, 3-MA and E-64d) or an equivalent volume of DMSO (mock control). HA, hemagglutinin tag. Red dots indicate protein bands of correct size.

FIG. 4—RPN10 homologues are conserved among plants and animals. (A) Phylogenetic analysis of RPN10 proteins from various organisms. The presence of vWA and UIM domains were predicted by PFAM. AtRPN10, Arabidopsis thaliana RPN10 (Uniprot ID: P55034); SI RPN 10, Solanum lycopersicum RPN10 (A0A3Q7F6N7); OsRPN10, Oryza sativa RPN10 (O82143); ZmRPN10, Zea mays RPN10 (B6TK61); DmRPN10, Drosophila melanogaster RPN10 (P55035); HsRPN10, Homo sapiens RPN10 (Q5VWC4); BtRPN 10, Bemisia tabaci RPN10 (XP_018915695); MqRPN10, Macrosteles quadrilineatus RPN10; MpRPN10, Myzus persicae RPN10 (XP_022181722.1). (B) Sequence alignment of the A. thaliana RPN10 and the M. quadrilineatus RPN10 proteins. The vWA domains and UIM domains are highlighted in red and blue, respectively.

FIG. 5—SAP05, ZnF domain of SPL5 and the vWA domain of AtRPN10 form a ternary complex in vitro. (A). Recombinant SAP05 and the ZnF domain of AtSPL5 form complex in gel filtration chromatography. Upper panel: Gel filtration chromatography of SAP05, ZnF domain of SPL5 and the SAP05-ZnF_SPL5 complex. Lower panel: Coomassie staining of corresponding protein in different elution fractions. (B). The vWA domain of AtRPN10 and the ZnF domain of AtSPL5 were co-purified with a his-tagged SAP05 when co-expressed in E. coli. His-tagged protein were purified by immobilized metal affinity chromatography (iMAC). vWA and evWA (a two-amino-acid point mutation of vWA that showed decreased binding with SAP05 in yeast two-hybrid assays) were tagged with a sumo tag to increase protein solubility. L: ladder. T: total cell extract. S: soluble fraction. P: protein purified by iMAC. (C). The vWA domain of AtRPN10, SAP05 and the ZnF domain of AtSPL5 form a ternary complex in the His-tag pull-down assay. His-sumo-tagged vWA domain or vWA domain were used as bait in iMAC to pull-down untagged SAP05 and/or ZnF domain of AtSPL5

FIG. 6—Residues on SAP05 homologs that might determine effector binding specificity. The alignment of SAP05 homologs are shown, The names of phytoplasma strains are labelled in the left side, with colours corresponding to SPL or GATA binding specificity. Blue: GATA-binding only; Red: SPL-binding only; Black: GATA- and SPL-binding. Residues boxed in red are likely to be involved in SPL or GATA binding.

FIG. 7—SAP05 can mediate protein degradation in human 26S proteasome. Purified human 26S proteasomes degrade His-SPL5 in the presence of SAP05 and A. thaliana vWA, and MG132 inhibits this degradation. Western blots shown are from protein extracts of recombinant human 26S proteasome preparations (BostonBiochem) in the presence of purified His-SPL5 and SAP05 with or without A. thaliana RPN10 vWA (AtvWA) or proteasome inhibitor MG132 probed with antibodies to HA, GFP and SAP05 as shown at left. Red dots at left of the blots indicate the expected sizes of TFs. Protein loading was visualized using Amido black staining.

FIG. 8—Insect directed engineering of A. thaliana RPN10 confers resistance to SAP05 action. (A) Schematic overviews of domain organizations of A. thaliana and M. quadrilineatus RPN10 proteins and alignment of the first 70 residues of plant and animal RPN10 vWA domains. Highly divergent residues are highlighted below the alignment. (B) Specific residues within the A. thaliana RPN10 vWA domain are required for SAP05 interaction in Y2H assays. Interaction of the AtRAD23B proteasome shuttle factor with AtRPN10 is included as a control to show that RPN10 is functional as an interactor in yeast. (C) Specific residues within the A. thaliana RPN10 vWA domain are required for SAP05 degradation of plant GATA and SPL in A. thaliana protoplasts.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

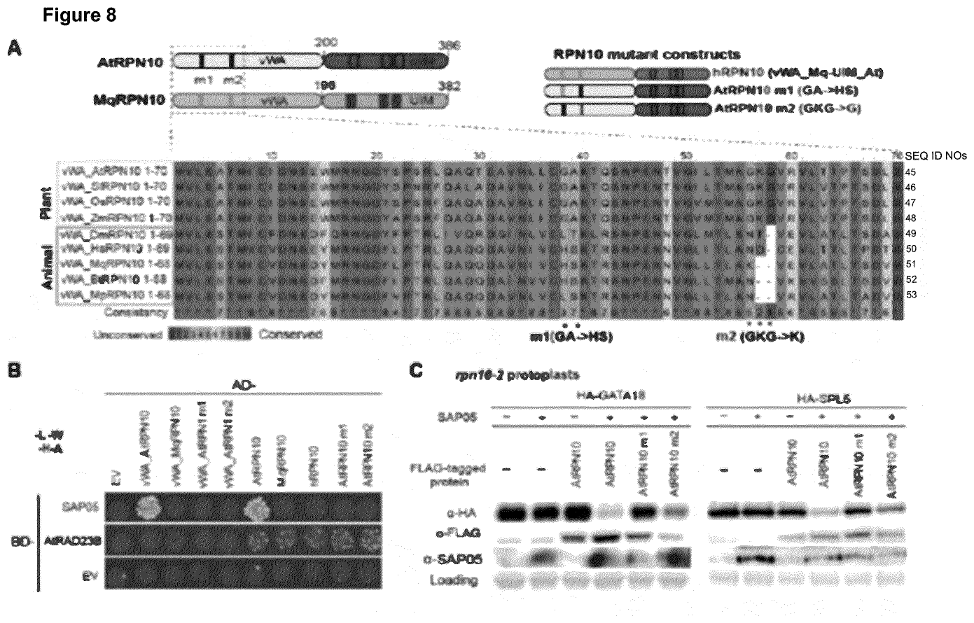

RPN10 is a subunit of the 26S proteasome that recognises polyubiquitinated proteins. RPN10 is characterised by an N-terminal von Willebrand factor type A domain (vWA) and a C-terminal domain containing Ubiquitin-interacting motifs (UM). We have found that the Phytoplasma protein SAP05 protein interacts specifically with the vWA domain of RPN10, and furthermore, that the vWA domain is all that is needed to mediate protein degradation. Specifically, we have found that the HS (human RPN10) or GA (plant RPN10) residues in vWA are a key binding region for SAP05. This is shown in FIG. 8. We further show that mutation of these residues prevents SAP05 binding and therefore SAP05-mediated protein degradation.

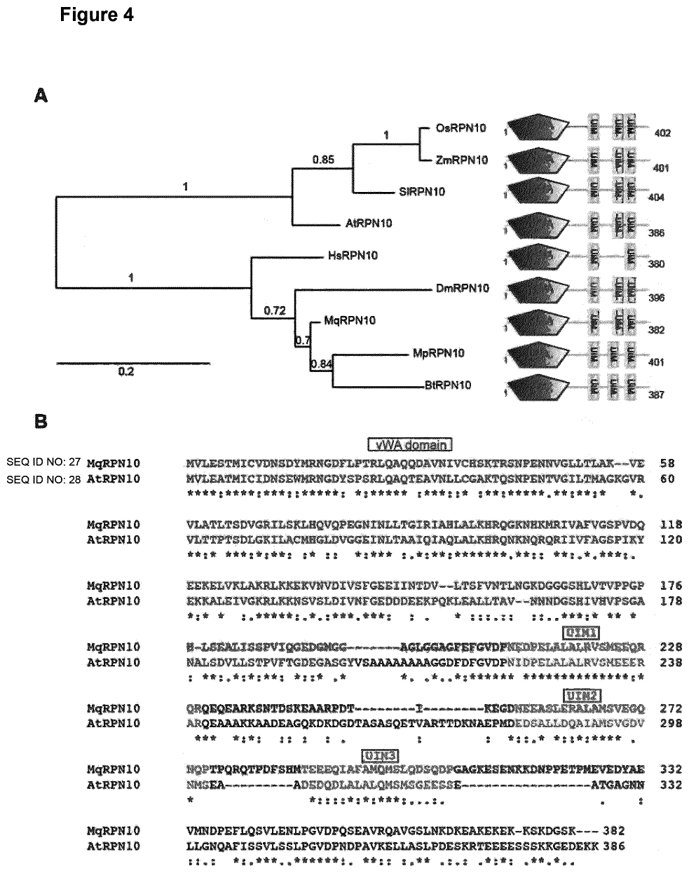

We have found that SAP05 interacts with RPN10 (and specifically the vWA domain thereof) to induce ubiquitin-independent protein degradation. This interaction provides an opportunity to harness this natural mechanism for the purpose of selectively degrading target proteins via the proteasome in a ubiquitin-independent manner. As shown in FIG. 4, RPN10 is highly conserved across eukaryotes, meaning that this mechanism is applicable to, and thus will be highly useful across, many organisms. The present invention can also be used to selectively degrade target proteins in host cells, tissues or whole organisms.

Accordingly, in a first aspect of the invention, there is provided a fusion protein comprising a vWA targeting moiety or a variant thereof and a target-binding moiety. In some embodiments, the vWA targeting moiety specifically recognises the HS or GA residue of vWA or the region of vWA that is on the opposite site of the area of vWA that binds the 26S proteasome. The HS residue of vWA may be the amino acids at positions 38 and 39 of SEQ ID NO: 24 or a corresponding position in a homologous sequence, or the amino acids at positions 38 and 39 of SEQ ID NO: 26 or a corresponding position in a homologous sequence. The GA residue of vWA may be the amino acids at positions 38 and 39 of SEQ ID NO: 5, or amino acids at positions 38 and 39 of SEQ ID NO: 21 or a corresponding position in a homologous sequence.

Preferably, the vWA targeting moiety is a SAP05 peptide.

In a further embodiment, the fusion protein may further comprise a RPN10 protein or a VWA domain. In an alternative embodiment of the methods and uses described herein a fusion protein comprising a vWA targeting moiety and a target-binding moiety may be administered in combination with a (separate) RPN10 peptide or vWA domain peptide. Preferably, RPN10 or vWA is administered concurrently with the fusion protein or SAP05 protein.

By “fusion protein” is meant a protein that comprises portions (for example, subunits, motifs or domains) from two or more proteins; for example a fusion protein may comprise a SAP05 protein and an immunoglobulin domain. Alternatively, the fusion protein may comprise a bispecific antibody or nanobody (i.e. an antibody with two binding specificities) which can recognise both a vWA domain, or portion thereof and a target protein. The two or more proteins are typically heterologous proteins, but in some circumstances a fusion protein may comprise multiple portions from the same protein. A fusion protein may be encoded by a nucleic acid generated through the joining of two or more genes or motifs or domains from genes that originally coded for separate proteins. Fusion proteins are also known as chimeric proteins.

By “target-binding moiety” is meant any structure capable of binding to a target protein or a portion of fragment of a target protein. This includes peptides, cyclic peptides, binding domains, small molecules/chemicals, t-cell receptors, antibodies, functional fragment of an antibody, nanobodies, or aptamers. In one embodiment, the target-binding moiety is a modified PROTAC, preferably wherein the E3 ligase portion of the PROTAC is replaced with SAP05. A particular advantage of this fusion protein is that neither SAP05 nor PROTAC is degraded with the target protein and thus the fusion protein can be recycled for further cycles of target protein degradation.

By “vWA targeting moiety” is meant any structure capable of binding to a vWA domain for example, as shown in SEQ ID NO: 21 or a homologue thereof, or portion or fragment of the vWA domain, and in particular a portion of fragment that is not involved in binding to the proteasome. Such a targeting or binding moiety may include peptides, cyclic peptides, binding domains, small molecules/chemicals, t-cell receptors, antibodies, functional fragment of an antibody, nanobodies, or aptamers. In one embodiment, a vWA targeting moiety binds at least the GA or HS residues at positions 38 and 39 of SEQ ID NO: 2, 21, 24 or corresponding residues in homologous sequences.

Suitable homologues can be identified by sequence comparisons and identification of conserved domains using databases such as NCBI and Plant ensemble and alignment programmes known to the skilled person. Thus, one of skill in the art will be able to identify the analogous amino acid substitutions listed above with reference to SEQ ID NO: 2, 21, 24 or 26 by aligning SEQ ID NO: 2, 21, 24 or 26 with the candidate homologous sequence. Thus, the nucleotide sequences of the invention and described herein can be used to isolate corresponding sequences from other organisms.

In this manner, methods such as PCR, hybridization, and the like can be used to identify such sequences based on their sequence homology to the sequences described herein. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Thus, for example, probes for hybridization can be made by labelling synthetic oligonucleotides based on the sequences of the invention. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

The term “variant” or “functional variant” as used herein with reference to SAP05 or RPN10 or a vWA domain as defined herein refers to a variant gene sequence or part of the gene sequence which retains the biological function of the full non-variant sequence. That is, a SAP05 variant is able to bind RPN10, subsequently leading to degradation of a target protein. Similarly, a vWA domain variant is able to bind to SAP05. A functional variant also comprises a variant of the SAP05 or RPN10 gene, which has sequence alterations that do not affect this function, for example in non-conserved residues. Also encompassed is a variant that is substantially identical, i.e. has only some sequence variations, for example in non-conserved residues, compared to the wild type sequence as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence, for example as defined in SEQ ID NO 1 or 19. In some embodiments, SAP05 comprises an amino acid sequence as defined in SEQ ID NO: 1 or a functional variant thereof. In some embodiments, SAP05 consists of the amino acid sequence as defined in SEQ ID NO: 1 or a functional variant thereof.

In one embodiment, the variant of SAP05 comprises one or more sequence variations (or mutations, such terms may be used interchangeably) from SEQ ID NO: 1. Such sequence variations preferably prevent or reduce binding of SAP05 to its endogenous targets, such as the Zn-finger proteins, GATA and SPL. In one embodiment, the variant SAP05 comprises one or more mutations in SEQ ID NO:1 in the regions responsible for binding GATA and/or SPL. Preferably, such a mutation may be selected from one or more of the following positions in SEQ ID NO: 1: 41D, 42M, 67F, 68T, 73R, 83M and 84I. The mutation may be a deletion or a substitution at one or more of these positions, but preferably the mutation reduces or abolishes binding of SAP05 to GATA and/or SPL transcription factors.

In another embodiment, the variant of SAP05 may be selected from one or more of the following sequences: SEQ ID NO: 7, 8, 9, 10, 11, 12, 13, 14, 15, 16 and 17. SAP05_AYWB binds both GATAs and SPLs; _ATP, SAP05_WBDLa, SAP05_PnWBa bind only SPLs;WBDLb and SAP05_PnWBb bind only GATAs.

In some embodiments, the protein further comprises a linker, linking the vWA targeting moiety and target-binding moiety and/or linking the vWA targeting moiety and the vWA domain. The linker may be cleavable or non-cleavable. An example of a linker is a GS linker.

In further embodiments, the fusion protein is for use as a medicament. By “medicament” is meant any substance used for medical treatment.

In another aspect of the invention, there is provided an expression vector comprising a nucleic acid encoding the fusion peptide, or a nucleic acid comprising the nucleic acid sequence as defined in SEQ ID NO: 2 or a functional variant thereof. In a preferred embodiment, the nucleic acid sequence encoding the fusion protein is operably linked to a regulatory sequence such as a promoter, as described below.

Suitable vectors for the expression of the fusion protein can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. Vectors may comprise an inducible promoter, so that the vector (and therefore the fusion protein, and in some embodiments the RPN10 protein in some cases) is expressed only in certain conditions. This can allow for the vector only to be expressed in certain cells or tissues. For example, inducible promoters can be controlled by chemicals or environmental stimuli. Vectors may alternatively comprise a tissue specific promoter so that the expression occurs in specific cell or tissue types.

In some embodiments, the expression vector further comprises a nucleic acid encoding a plant RPN10 protein or a fragment thereof, preferably wherein the nucleic acid comprises the nucleic acid sequence as defined in SEQ ID NO: 6 or a functional variant thereof. In a preferred embodiment, the RPN10 fragment comprises or consists of the vWA domain. Accordingly, in one embodiment, the expression construct further comprises a nucleic acid encoding a vWA domain as defined in SEQ ID NO: 21 or a functional variant thereof. In a further embodiment, the expression construct further comprises a nucleic acid encoding a vWA domain wherein the nucleic acid sequence comprises SEQ ID NO: 22 or a functional variant thereof. In an alternative embodiment of the methods and uses described herein, RPN10 or vWA are expressed from a second separate expression construct to the SAP05 expression vector. Preferably, the RPN10 or wVA domain comprises a GA domain at residues 38 and 39 of the amino acid sequence (e.g. as is the case for plant RPN10 in SEQ ID NO: 5 or 21), rather than a HS domain at residues 38 and 39 of the amino acid sequence (e.g. as is the case of human RPN10 in SEQ ID NOs: 24 or 26).

Alternatively, a non-plant RPN10, such as a human RPN10 (e.g. those defined in SEQ ID NO: 24 and 26), can be modified so that the HS domain at residues 38 and 39 of the amino acid sequence is replaced with a GA domain (as is found in plant RPN10, such as that defined in SEQ ID NO: 5). For example, a human RPN10 as defined in SEQ ID NO: 24 and 26 can be edited using gene editing to replace “CATTCA” at positions 112 to 117 with “GGAGCC” (e.g. as at positions 112 to 117 of SEQ ID NO: 5). A non-plant RPN10 with such a modification may also be overexpressed in a cell or tissue as part of the invention.

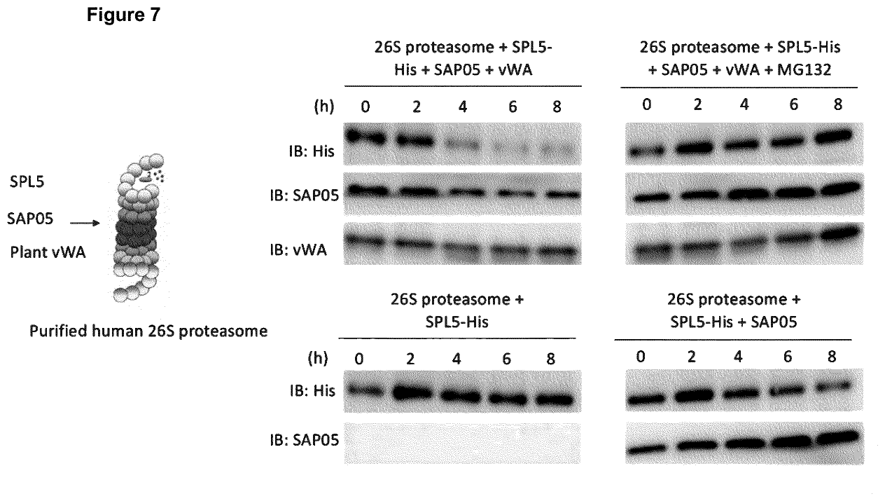

This is particularly helpful in applications using non-plant cells or organisms, for example, such as animals, including humans. Accordingly, in one embodiment, there is provided a method of targeted protein degradation in humans and animals. For example, as shown in FIG. 7, co-expression of SAP05 and a vWA domain can be used to degrade proteins through the human 26S proteasome. Having to deliver plant RPN10 together with SAP05 may increase the safety of any therapeutic application if leaky production of SAP05 in non-target tissues has lethal consequences. As a result, having to deliver ‘plant’ RPN10, or a modified non-plant RPN10 (e.g. a human RPN10, such as defined in SEQ ID NO: 23 or 25) with changes in amino acids in the region of vWA that is on the opposite site of the area of vWA that binds the 26S proteasome and that includes the GA amino acidsat residues 38 and 39 of the amino acid sequence of RPN10, with SAP05 will allow for engineering higher specificity.

In a preferred embodiment of any aspect of the invention, the plant RPN10 protein is derived from Arabidopsis thaliana or the protein comprises the amino acid sequence of SEQ ID NO: 5. In a preferred embodiment of any aspect of the invention, the plant vWA domain is derived from Arabidopsis thaliana or the protein comprises the amino acid sequence of SEQ ID NO: 21.

To allow two proteins to be expressed as individual proteins from a single mRNA molecule, ribosomal skipping sequences may be added to the 5′ and/or 3′ end of the nucleic acid encoding the fusion protein and/or nucleic acid encoding the plant RPN10 or vWA protein. During translation, when the ribosome encounters a ribosomal skipping sequence it is prevented from creating the peptide bond with the last proline in the ribosomal skipping sequence. As a result, translation is stopped, the nascent polypeptide released and translation is re-initiated to produce a second polypeptide. This results in the addition of a C-terminal ribosomal skipping sequence (or the majority of such a sequence) to the first polypeptide chain, and a N-terminal proline to the next polypeptide.

Accordingly, in a further embodiment, the nucleic acid construct comprises at least one ribosomal skipping sequence.

In one example, the ribosomal skipping sequence may be selected from one of the following:

F2A; A 2A DNA sequence variant used between two CDS.

F2A:
(SEQ ID NO: 7)
GGACAACTTCTCAACTTTGACTTGCTAAAGTTAGCTGGTGATGTTGAAT
CTAATCCTGGACCA

Use of the F2A sequence results in the addition of the F2Aaa1-20 polypeptide sequence to the C-terminus of the protein upstream of the ribosomal skipping site and a proline residue (F2Aaa21) to the downstream protein.

F2Aaa1-20:
(SEQ ID NO: 8)
GQLLNFDLLKLAGDVESNPG
F2Aaa21:
P

F2A30; A 2A DNA sequence variant used between two CDS.

F2A30:
(SEQ ID NO: 9)
CACAAACAGAAAATTGTGGCACCGGTGAAGCAGACTCTCAACTTTGACT
TGCTAAAGTTAGCTGGTGATGTTGAATCTAATCCTGGACCA

Use of the F2A30 sequence results in the addition of the F2A30aa1-29 polypeptide sequence to the C-terminus of the protein upstream of the ribosomal skipping site and a proline residue (F2A30aa30) to the downstream protein.

F2Aaa1-20:
(SEQ ID NO: 10)
HKQKIVAPVKQTLNFDLLKLAGDVESNPG
F2Aaa21:
P

In one embodiment, the nucleic acid encoding the fusion protein includes a C-terminal skipping sequence, preferably F2A30(aa1-29).

In a further embodiment, the nucleic acid encoding the plant RPN10 or vWA domain includes a N-terminal skipping sequence and F2A30(aa30), i.e. a proline amino acid residue.

In a further alternative embodiment, an internal ribosomal entry site (IRES), tRNA sequence, a ribozyme (such as a Hammerhead (HH) ribozyme unit and/or a hepatitis delta virus (HDV) ribozyme unit) or direct repeat (DR) sequence could be used instead of a ribosomal skipping sequence. Again, such sequences may be added to the 5′ and/or 3′ end of the nucleic acid encoding the fusion protein and/or the nucleic acid encoding the plant RPN10 or vWA protein and allows two proteins to be expressed as individual proteins from a single mRNA transcript and from a single regulatory sequence (promoter).

In a further aspect of the invention, there is provided a cell comprising the fusion protein or the expression vector described above. The cell is preferably a eukaryotic cell. Preferably the cell is a mammalian or plant cell. In some embodiments the cell may be a fungal or yeast cell.

In another aspect of the invention, there is provided a transgenic organism expressing the expression vector or comprising the cell described above, wherein the organism is not a human. The organism is preferably eukaryotic. The organism may be a mammal (but not a human) or a plant. Where the organism is a plant, the invention also covers seeds obtained or obtainable from the plant, as well as progeny obtained from the seed, and further generations of progeny. Preferably the seed and progeny comprise and express the expression vector. Where the organism is a mammal, the mammal may be for example a rodent (such as a mouse, rat), a lagomorph (such as a rabbit), a primate (such as a monkey for instance Macaca spp). In other embodiments the mammal may be a bovid, caprid, ovid, porcid. In other embodiments the organism may be avian; for example, a galloanserine such as chicken, duck. In one embodiment, the expression vector is stably incorporated into the organism's genome.

In another aspect of the invention, there is provided a genetically altered organism, wherein the organism comprises and expresses the expression vector or the fusion protein of the present invention. In one embodiment, the expression vector or the nucleic acid encoding the fusion protein is stably incorporated into the genome. This may be achieved using genome editing techniques such as CRISPR.

In a further aspect of the invention, there is provided the use of the fusion protein or expression construct of the invention in targeted protein degradation. By “targeted protein degradation” is meant reduction or elimination of levels of a target protein. Reduction may mean a decrease of at least 50%, 60%, 70%, 80%, 90%, 95% or 99% of the protein level before use of the fusion protein. Elimination means that the protein level is reduced to an undetectable level.

In some embodiments, the target protein degradation is ubiquitin-independent protein degradation, meaning that the protein degradation occurs without the need for ubiquitination.

In another aspect of the invention, there is provided a method of targeted protein degradation, the method comprising applying or administering the fusion protein described above or a vWA targeting moiety, or an SAP05 protein or the expression vector described above to an organism or to a sample. In an alternative embodiment, the method comprises applying a SAP05 (or another vWA targeting moiety) and a vWA protein as described above to an organism or to a sample. In a further aspect of the invention, there is provided a method of controlling the (protein) level of a target protein, the method comprising applying the fusion protein described above or a vWA targeting moiety or an SAP05 protein or the expression vector described above to an organism or to a sample. In another alternative embodiment, there is provided a method of controlling the (protein) level of a target protein, the method comprising applying a SAP05 (or another vWA targeting moiety) and a vWA protein as described above to an organism or to a sample.

The sample can be any form of sample that contains the target protein, for example, it may be a cell culture or a cellular sample derived from an organism or tissue. For example, where the organism is a plant, the sample may be a protoplast sample. In another example where the organism is a human, the sample may be a blood sample (or another other bodily sample). Applying or administering (such terms may be used interchangeably) may comprise simply adding the fusion protein or expression vector to the sample, but may also require one or more extra steps to deliver the fusion protein or expression vector into a cell. For example, it may be necessary to carry out methods such as electroporation, transfection, viral particle transfer (such as that disclosed in US 2012/0015899), or gene gun to cause uptake of the fusion protein or expression vector into a cell.

Controlling may mean reducing the level of a target protein to a level found in a steady state or a healthy state, controlling may also mean eliminating levels of a certain protein.

In one embodiment of any of the methods described above a fluorescently tagged antibody (or other target binding protein or fragment thereof) may be first applied to the sample, where the antibody is specific to the target protein. As such, application of the fluorescently tagged antibody labels the target protein. In this embodiment, the target-binding moiety of the fusion protein is specific for the fluorescent tag. As a result, degradation of the labelled target protein by SAP05 (or another vWA targeting moiety) in the sample can be detected by a decrease in fluorescent signal. This method allows a target protein to be detected and visualised using a fluorescently tagged antibody (e.g. with GFP), and then the degradation of the target protein to be detected and visualised using an fusion protein of the invention that specifically targets this fluorescent tag. Such a fusion protein can therefore be used to target any protein that is bound to a fluorescent antibody (or other target-binding protein). In other words, a single fusion protein can be used in combination with any other tagged target-binding protein providing a valuable research tool. Suitable fluorescent tags include GFP, YFP, CFP, or BFP, and any other fluorescent tag known to the skilled person. Accordingly, in a further aspect of the invention, there is provided a kit comprising a tagged target binding protein (e.g. an antibody), preferably a fluorescently tagged antibody and a fusion protein as described above, wherein the target-binding moiety of the fusion protein is specific for the tag.

In some embodiments of any of the methods described above, the target protein is a cytosolic protein. By “cytosolic protein” is meant any protein that is present in the cytosol. Examples of cytosolic proteins include hormones, cytokines, signalling proteins, transcription factors, structural proteins, effector proteins and enzymes, but many other examples will be known by the skilled person. In another embodiment, the protein may be an antibody; for example, an autoantibody.

In one embodiment, the target may be a SPL protein. Examples of SPL proteins include, but not are limited to, teosinte glume architecture1 (tga1) (that results in naked kernels in maize (Wang et al., 2005)), Ideal Plant Architecture 1 (IPA1), LG1, and GW8 and GLW7 (all of which encode in rice SPL proteins that were incorporated to breeding processes for improving crop traits (Ishii et al., 2013; Jiao et al., 2010; Miura et al., 2010; Si et al., 2016; Wang et al., 2018a; Wang et al., 2012)). In this embodiment, where the target protein is a SPL protein, the method is preferably the administration of a SAP05 protein (or another vWA targeting moiety).

In a further embodiment, the target may be a specific SPL protein, in that it may be a species-specific SPL protein, a specific member of an SPL family, or a product of a specific homoallele for an SPL protein. In this particular embodiment, the method may use a SAP05 variant protein which has reduced or abolished binding to its endogenous targets (compared with wild-type SAP05). The endogenous targets may include the Zn-finger proteins GATA and SPL as described above. The SAP05 variant protein is fused to a target binding moiety which is specific for the specific SPL protein to form a SAP05 fusion protein.

In some embodiments, the target protein is a nuclear protein, such as but not limited to histone proteins. We believe that, in general, SAP05 fusion proteins will be sufficiently small to migrate into the cell nuclei, and the 26S proteasome is also active within the nuclei.

In alternative embodiments, the target protein is a cell surface protein. By “cell surface protein” is meant any protein that is present on the cell surface. Cell surface proteins often play a vital role in the communication between the cell and its environment. Examples of cell surface protein include receptors, transporters, channels, and cell-adhesion proteins, but many other examples will be known by the skilled person. Preferably, in this embodiment the SAP05 protein, the vWA targeting moiety or fusion protein comprises a localisation signal that causes such a protein to be located to the endoplasmic reticulum and/or golgi apparatus. A suitable localisation signal includes KKXX (SEQ ID NO: 18) or KDEL (SEQ ID NO: 19). Alternatively, the vWA targeting moiety or SAP05 may be able to mediate degradation of cell surface proteins through an interaction between RPN10 or vWA and the autophagosome (a component of the autophagy pathway).

In another embodiment, the target protein is a species-specific protein, the targeting of which for degradation by the methods or fusion proteins of the invention can be used as a pesticide including insecticides, herbicides, fungicides and nematicides. Examples include proteins involved in producing the chitin skeletons of insects, cytochrome P450 proteins that are involved in insecticide resistance and degradation of plant defence molecules, proteins involved in spore and appressorium development (and plant infection organelles) of fungi and oomycetes, specific KAI proteins to controls weeds (e.g. https://www.biorxiv.org/content/10.1101/376939v2) and proteins involved in mucus production of slugs.

In other embodiments, the target protein is an exogenous protein, i.e. a protein that has been introduced into a cell or organism that is not usually expressed by that cell or organism. In one example, this exogenous protein is a CRISPR enzyme, such as Cas or Cpf1. In one example, Cas nuclease from a CRISPR-Cas system that has been introduced into a cell or organism, such as Cas9, can be selectively or conditionally degraded by the fusion protein or expression vector of the invention to limit off-target effects and/or unwanted persistence of the CRISPR-Cas system once the desired gene editing has been obtained. In particular, where the expression vector of the invention is operably linked to an inducible or tissue-specific promoter, targeted degradation of the CRISPR enzyme can be controlled either spatially or temporally. In one example, this may be desirable where gene knock-out at the embryonic stage or at a certain developmental stage would be lethal.

Targeted genome modification or targeted genome editing is a genome engineering technique that uses targeted DNA double-strand breaks (DSBs) to stimulate genome editing through homologous recombination (HR)-mediated recombination events.

Type II CRISPR is one of the most well characterized systems and carries out targeted DNA double-strand breaks in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. The specificity of the system can be programmed by selecting appropriate spacer sequences; for ease of use, the tracrRNA, spacer, and crRNA regions can be combined into a single RNA molecule, referred to as a single guide RNA or sgRNA.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Accordingly, in a further aspect of the invention, there is provided a method of gene editing, the method comprising introducing a CRISPR-Cas system comprising a CRISPR enzyme to a cell or organism, allowing the CRISPR-Cas system to edit a gene, and delivering a fusion protein according to the invention comprising a target moiety that is specific for said CRISPR enzyme, and allowing the fusion protein to degrade the CRISPR enzyme so as to inhibit any further activity of the CRISPR-Cas system. The fusion protein may be delivered together with the CRISPR-Cas system, or separately; and may be delivered simultaneously or sequentially; depending on the desired timing of cleavage and degradation.

In other embodiments of any of the methods described above, the target protein can cause pathology in a target organism, and/or the target protein may be a drug target.

In another aspect of the invention, there is provided a method of modulating a biochemical or physiological response in an organism, the method comprising administering the fusion protein or the expression vector described above. Modulating may mean increasing or decreasing depending on the desired outcome. For example, there are situations where an increase in immune response is desired (e.g. to boost the immune response against cancer) or a decrease in immune response is desired (e.g. to limit the immune response during auto-immunity). In this example, it would be clear to the skilled person that a response can be increased by degrading an inhibitory protein, and a response can be decreased by degrading a stimulatory or effector protein. There may also be an interest in increasing or decreasing a physiological response in research to determine the effect of such a response in a cell, tissue or organism. In some embodiments, the physiological response is selected from a stress response, an immune response, a hormone response, chemical response or a light response.

A stress response is the response of a cell or organism to a stressor such as an environment condition. A stressor may be abiotic or biotic. An“abiotic stressor” includes drought, salinity, wind, high or low temperature or high light. A “biotic stressor” refers to harmful effects caused by another (living) organism such as by the secretion of a toxin or an effector. Accordingly, in one embodiment, the method increases the resistance of an organism to a stressor.

An immune response is the response of an organism to a foreign or harmful antigen. In some cases, it is desirable to increase the immune response, for example in the context of cancer or infection where there is a suboptimal immune response raised which causes pathology. In other cases, it is desirable to decrease immune response, for example in the context of autoimmunity or a cytokine storm, where self-recognition or and/or excessive responses by the immune system can cause inflammation and damage to the host. In certain embodiments, the target-binding moiety is specific against an autoantibody such as those raised in autoimmune conditions like rheumatoid arthritis, multiple sclerosis, type 1 diabetes, lupus, inflammatory bowel disease or psoriasis and related conditions, or in infections such as anti-IFN antibodies during SARS-CoV-2 infection.

A hormone response is the response of a cell or organism to a hormone, wherein the hormone may be secreted by another cell or organism and is sensed by the cell or organism of the invention. Hormones may be eicosanoids, steroids or amino acid/protein derivatives. Hormones may affect a variety of processes including digestion, metabolism, respiration, tissue function, sensory perception, sleep, excretion, lactation, stress induction, growth and development, movement, reproduction and mood.

A chemical response is the response of a cell or organism to a chemical, such as to a toxin or volatile compound.

A light response is a response of a cell or organism to a light stimulus, and this may be a light stimulus of a specific wavelength. A light response includes phototropism, which is the growth of an organism in response to a light stimulus, and photoperiodism, which is the flowering or change in other development processes in response to the photoperiod (i.e. the length of time a light stimulus is received). A light response may cause an effect on circadian rhythm.

In a further aspect of the invention, there is provided a method of treating a condition in a patient in need thereof, the method comprising administering the fusion protein or the expression vector described above. The condition may be any condition that is characterised or caused by the expression or increased expression of a protein that causes pathology in the host organism.

In another aspect of the invention, there is provided the fusion protein or expression vector of the invention for use in the treatment of a condition that is characterised or caused by the expression or increased expression of a protein that causes pathology in the host organism. In a further aspect of the invention, there is provided the use of the fusion protein or expression vector of the invention in the manufacture of a medicament for the treatment of a condition that is characterised or caused by the expression or increased expression of a protein that causes pathology in the host organism.

As used herein, increased expression refers to a level of expression that is higher than the level of expression in a host organism that lacks the given pathology.

In one embodiment, the condition may be cancer, infection, a neurodegenerative disorder and/or a proteopathy. Alternatively, the condition may be an autoimmune disease or a metabolic bone disease.

The cancer may be a carcinoma, sarcoma, lymphoma, leukemia, germ cell tumour or blastoma. Examples of cancer include bone and muscle sarcomas, brain and nervous system cancers, breast cancers, endrocrine system cancers, eye cancer, gastrointestinal cancer, genitourinary and gynecologic cancers, head and neck cancers, hematopoietic cancers, skin cancers, thoracic and respiratory cancers. Other specific cancers will be known to the skilled person. Where the condition is a cancer, the binding moiety of the fusion protein may be specific for an oncogene protein, a growth factor, an extracellular matrix protein, a cytoskeleton protein, a cell cycle protein, a checkpoint protein, or a receptor.

Examples of infection include infection with viruses, viroids, bacteria, fungi, prions, parasites and arthropods. An infection may include an antibiotic-resistant bacterial infection, a hospital acquired infection, a superinfection, or a biofilm; which are all recognised as difficult to treat.

Examples of neurodegenerative disorders include amyotrophic lateral sclerosis, Parkinson's disease, Alzheimer's disease, fatal familial insomnia, and Huntington's disease.

Proteopathies are also known as protein conformational disorders or protein misfolding diseases, and cause disease due to proteins becoming structurally abnormal leading to toxicity or lack of normal function of the protein. Examples of proteopathies include: Creutzfeld-Jakob disease and other prion diseases, Alzhemier's disease, Parkinson's disease, amyloidosis, multiple system atrophy, cystic fibrosis, tauopathies and other aggregate-prone disorders, amyotrophic lateral sclerosis, type II diabetes, and sickle cell disease. Where the condition is a proteopathy, the binding moiety of the fusion protein may be specific for tau or beta-amyloid.

In some embodiments, the condition is characterised by increased expression or activity of a target protein, and the target-binding moiety is specific for said target protein.

In another aspect of the invention, there is provided a method of treating an infection caused by a microorganism, the method comprising administering the fusion protein or the expression vector described above, wherein the target binding moiety is specific for a protein expressed by a microorganism. By “microorganism” is meant a bacteria, archaea, fungi, protozoa, algae, nematodes or virus.

In some embodiments, the microorganism is pathogenic (i.e. is a pathogen). In certain embodiments, the target protein is a protein that is essential for the survival and/or replication of the microorganism, and therefore degrading this protein causes death or non-replication of the microorganism. In other embodiments, the target protein is an effector molecule expressed by the microorganism (i.e. a molecule that causes pathology). In further embodiments, this effector molecule is a microbial toxin, and preferably the method is treating an infection which has caused sepsis or toxic shock in a subject. Microbial toxins may be produced by gram-positive or gram-negative bacteria, or fungi, and examples include cholera toxin, diphtheria toxin, pertussis toxin, mycoplasma toxin, E. coli toxin, Shiga toxin, Pseudomonas exotoxin A, Botulinum toxin, Tetanus toxin, Anthrax toxin, Bordetella pertussis AC toxin, Bacillus anthracis EF toxin, S. aureus exofolitin B, perfringiolysin O, hemolysin, listeriolysin, alpha toxin, pneumolysin, streptolysin O, leucocidin and pyrogenic exotoxins.

In certain embodiments, a pathogen or pest excretes a toxin or an effector into the cytosol of cells. In this case, an expression vector comprising an inducible promoter which is only induced during infection with said pathogen drives expression of an expression vector containing a fusion protein according to the invention (and RPN10 or vWA in some cases) targeting a toxin or effector from said pathogen. Such pathogen inducible promoters include PPP1, hsr203J, gst1, PR1, PR5 promoters, and also includes synthetic pathogen inducible promoters. Therefore, specific fusion protein according to the invention (and optionally RPN10 or vWA) expression can be delivered only in cells and/or tissues affected by the pathogen.

In some embodiments, the pathogen or pest excretes a toxin or effector in a plant. Such pathogens or pests affecting include plant pathogenic bacteria like Pseudomonas, Xanthomonas, Ralstonia, Erwinia, Pantoea, Liberibacter, Xylella fastidiosa, Clavibacter, and Streptomyces; plant pathogenic fungi like Fusarium and Colletotrichum; oomycetes such as Phytophthora infestans, Phytophthora palmivora, Phytophthora ramorum, Phytophthora capsica, Pythium, and Powdery mildews; Protozoa like Plasmodiophora brassicae, and Polymyxa betae; nematodes like Potato cyst nematodes; pests like aphids, leafhoppers, psyllids, whiteflies, planthoppers, Phylloxera, mealybugs, sharpshooters and froghoppers.

In some embodiments, the pathogen or pest excretes a toxin or effector in a fish. Such pathogens or pests affecting fish include viruses like rhabdovirus, Esocid lymphosarcoma and Saprolegnia sp.

In a further aspect of the invention, there is provided a method of increasing the immunogenicity of a protein, the method comprising administering the fusion protein or the expression vector described above, wherein the target-binding moiety is specific for the protein, and wherein proteasome degradation of the protein results in increased antigen presentation of peptides degraded from the protein.

Immunogenicity is the ability of an antigen to trigger an immune response in a host. Degradation of proteins into shorter peptides allows for presentation of these peptides by antigen-presenting immune cells to the rest of the immune system in order to trigger an immune response against the antigen. Accordingly, this method can be particularly useful in the context of cancer or infection, where a sub-optimal immune response occurs due to a lack of immunogenicity to a protein. For example, protein degradation of cancer cell surface markers may allow for an improved cytotoxic T cell response against cancer cells expressing this marker due to increased immune recognition. Therefore, in some embodiments, the fusion protein or the expression vector described above is administered to a subject suffering from an infection or from cancer.

In another aspect of the invention there is provided a method for creating a protein knockout model, the method comprising administering the fusion protein or the expression vector described above to a cell or an organism. Such models may be used to study protein function in development, homeostasis and/or disease. In some embodiments, the model is a disease model and the disease is caused or characterised by a dysfunction or absence of a target protein, and the target moiety is specific for the target protein.

In a further aspect of the invention, there is provided a method of identifying a degradation effect of a target protein in a biological system, the method comprising applying the fusion protein described above, a vWA targeting moiety or a SAP05 (and optionally vWA) protein or the expression vector described above to the biological system. The biological system may be a cell, a tissue or an organism. The target protein may have many downstream effects. In some embodiments, where the target protein is a transcription factor or a regulatory protein, and the method further comprises performing RNA sequencing. By “regulatory protein” is meant any protein that controls the rate of transcription of DNA to RNA through specific DNA binding, and includes transcription factors.

RNA sequencing, also known as RNA-Seq, is a next-generation sequencing method that allows for measurement of RNA in a biological sample, known as the transcriptome. Methods for carrying out RNA sequencing are known to the skilled person and are well described in the art. RNA sequencing can be helpful in this context to identify the degradation effect of a target protein, particular of regulatory proteins, in a biological system, as RNA sequencing can be used before and after applying the fusion protein or expression vector described above and the results can be compared.

In another aspect of the invention, there is provided a method of increasing yield in a plant, the method comprising introducing the fusion protein or the expression vector described above to the plant, wherein the target-binding moiety is specific for a target protein the expression or activity of which is negatively correlated with yield. The plant may be selected from rice, maize, wheat, barley, sorghum, potato, tomato, cotton, soybean, Brassicas, such as B. napus, coconut, papaya, oil palms, grape, apple, oranges, sugarcane, citrus (such as lime, citrus, orange, grapefruit), egg plant, elm, ash, willow, elm, sesame, alfalfa, pea, plantain, birch, cassava, peanut, loofah, cocoa, date or date palm, sweet potato, lettuce, chrysanthemum, poinsettia, sunflower, phlox, hortensia, tulips, gladiolus and other bulbs, onion, garlic, cabbage tree, pine,trees, stone fruit trees, palm, carrot, strawberry, blueberry, cranberry and other berry plants. Alternatively, the plant may be a non-vascular plant, for example: liverworts (e.g. Marcanthia), mosses, hornworts, or verns. The term “plant” as used herein encompasses whole plants and progeny of the plants and plant parts, including seeds, fruit, shoots, stems, leaves, roots (including tubers), flowers, tissues and organs, wherein each of the aforementioned carry at least one of the herein described mutations. The term “plant” also encompasses plant cells, suspension cultures, callus tissue, embryos, meristematic regions, gametophytes, sporophytes, pollen and microspores, again wherein each of the aforementioned comprises the mutations as described herein.

The invention also extends to harvestable parts of a plant of the invention as described herein, including but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. In another aspect of the invention, there is provided a product derived from a plant as described herein or from a part thereof.

The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

Thus, according to the invention, yield comprises one or more of and can be measured by assessing one or more of: increased seed yield per plant, increased seed filling rate, increased number of filled seeds, increased harvest index, increased viability/germination efficiency, increased number or size or weight of seeds or pods or beans or grain, increased growth or increased branching, for example inflorescences with more branches, increased biomass, increased fresh weight or grain fill. Preferably, increased yield comprises at least one of an increased number or weight of seeds, beans or pods per plant, increased thousand kernel weight (TKW), increased biomass, increased fresh weight and increased growth. Yield is increased relative to a control or wild-type plant. For example, the yield is increased by 2%, 3%, 4%, 5%-50% or more compared to a control plant, for example by at least 10%, 15%, 20%, 25%, 30%, 35%, 40%, 45% or 50%.

In another aspect of the invention, there is provided a method of reducing or removing glutens in a plant, the method comprising introducing the fusion protein or the expression vector described above to the plant, wherein the target-binding moiety is specific for gliadin or glutenin. Gliadin is the water-soluble component of gluten and gliadin is the water-insoluble component of gluten. It is particularly advantageous to reduce or remove gliadins in a plant, as this is the principal toxic component of gluten for celiac patients.

Methods of the invention can also be used in fungi, for example to degrade a specific target protein which may be responsible for an unwanted effect arising from fungal growth, eg, food spoilage, infection, toxicity.

In some embodiments of any of the methods described above, the targeted protein degradation is ubiquitin-independent protein degradation as described above.

In a further aspect of the invention, there is provided a pharmaceutical composition comprising SAP05, a vWA targeting moiety, the fusion protein or the expression vector described above, and a pharmaceutically acceptable diluent, carrier or excipient. In some embodiments, the at least one pharmaceutically acceptable diluent, carrier or excipient is physiological saline, phosphate buffered saline (PBS) and/or sterile water. In some embodiments the pharmaceutical composition essentially consists of the fusion protein or the expression vector described above. In some embodiments, the pharmaceutical composition is for use in treatment of cancer, infection, a neurodegenerative disorder or proteopathy, as described above.

In another aspect of the invention there is provided a pharmaceutical composition for use in therapy. In another aspect of the invention there is provided a method of treating a condition, the method comprising administering the pharmaceutical composition of the invention to a patient in need thereof.

In another aspect of the invention, there is provided the pharmaceutical composition of the invention for use in the treatment of a condition that is characterised or caused by the expression or increased expression of a protein that causes pathology in a host organism. In a further aspect of the invention, there is provided the use of the pharmaceutical composition of the invention in the manufacture of a medicament for the treatment of a condition that is characterised or caused by the expression or increased expression of a protein that causes pathology in a host organism. In a further aspect of the invention there is provided a method of treating a condition that is characterised or caused by the expression or increased expression of a protein that causes pathology in a host organism, the method comprising administering the pharmaceutical composition of the invention to a patient in need thereof.

In one embodiment, the condition may be cancer, infection, a neurodegenerative disorder and/or a proteopathy. Alternatively, the condition may be an autoimmune disease or a metabolic bone disease.

In another aspect of the invention there is provided a kit comprising the pharmaceutical composition of the invention and preferably, instructions for use.

In another aspect of the invention, there is provided a screening library comprising a plurality of fusion proteins as described above, wherein each fusion protein comprises a different target-binding moiety.

In some embodiments, each target-binding moiety is specific for a different protein. This may be particularly helpful in drug discovery, whereby a variety of targets can be evaluated as binding moieties can be screened against a disease model (e.g. a cancer cell line) to find fusion proteins containing target-binding moieties that are effective in a given disease. In some embodiments the disease model may be a model organism (eg, a mouse), or may be a transgenic or non-transgenic cell line (e.g. a transgenic yeast or other fungal cell line, or a transgenic mammalian cell line) which expresses a protein of interest for screening.

In other embodiments, each target-binding moiety is specific for the same protein, but for a different epitope or binding region on the protein. Therefore, this screening library can be used to find the most effective target-binding moiety against a given target. This may be particularly helpful where e.g. there is need to screen a variety of antibodies against a known disease protein to find a fusion protein comprising the most effective antibody against the known disease protein.

Therefore, in a related aspect of the invention, there is provided a method for screening target-binding moieties using the screening library described above.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### EXAMPLE 1: SAP05 DEGRADES TARGET PROTEINS VIA RPN10

We have found that SAP05 interacts with the 26S proteasome subunit RPN10 in a Y2H screen. RPN10 is located within the 19S regulatory particle of the proteasome and serves as one of the main ubiquitin receptors recruiting ubiquitinated proteins for proteasomal degradation.

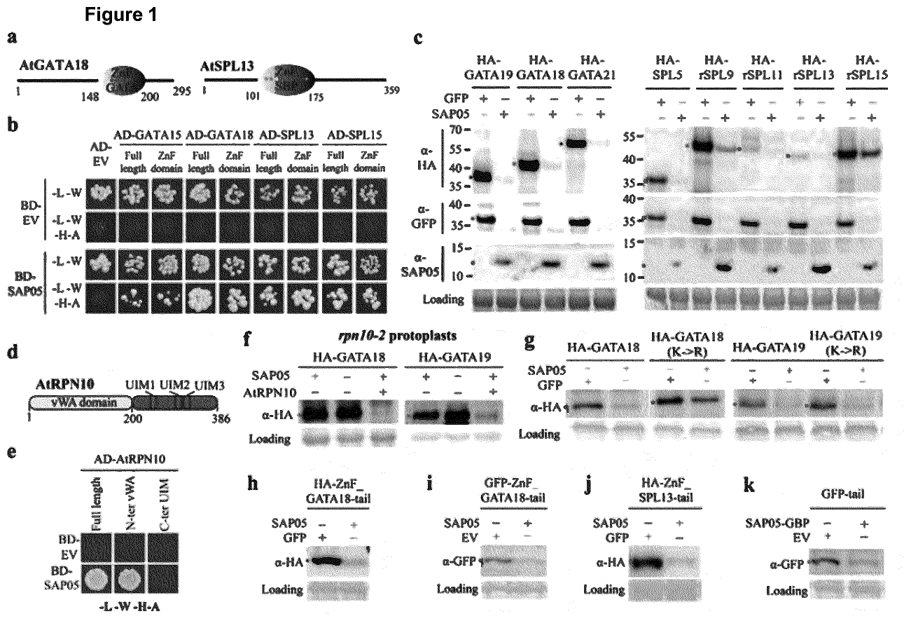

We also found that SAP05 interacts with several A. thaliana zinc-finger transcription factors, specifically GATAs and SPLs, in a yeast two-hybrid (Y2H) screen against an A. thaliana seedling library (FIG. 1A). Of the 29 GATA and 16 SPL family members in A. thaliana, 26 GATAs and 12 SPLs were successfully cloned and shown to interact with SAP05 in Y2H assays (FIG. 1B), indicating that SAP05 most likely binds all members of both families. SPLs regulate plant developmental phase transitions, and most are developmentally regulated by microRNA156 (miR156), whereas GATA proteins regulate photosynthetic processes, leaf development and flower organ development.

To investigate whether SAP05 degrades GATA and SPL transcription factors in plant cells, we transiently co-expressed several GATA or SPL genes with SAP05 (or GFP as control) in A. thaliana protoplasts. GATA proteins were absent or less abundant in the presence of SAP05 as compared to GFP (FIG. 10, left panel). Similarly, the abundances of five SPL proteins were visibly reduced in the presence of SAP05 (FIG. 10, right panel). Addition of MG132, a potent proteasome inhibitor, inhibited the SAP05-mediated destabilisation of two GATA proteins tested (FIG. 1I). Together, these data indicate that SAP05 degrades GATA and SPL transcription factors, via the 26S proteasome.

To investigate the role of RPN10 in the SAP05-mediated destabilisation of plant transcription factors, we made use of the existing and well-described A. thaliana loss-of-function rpn10 mutant line rpn10-2. In the protoplasts of rpn10-2 plants, SAP05 no longer degraded GATA18/HAN or GATA19, whereas when A. thaliana RPN10 was reintroduced into the protoplasts, these plant transcription factors were degraded (FIG. 1F). Therefore, RPN10 is required for the SAP05-mediated degradation of plant targets.

RPN10 is one of the primary ubiquitin receptor proteins in eukaryotes. RPN10 has two main domains, an N-terminal vWA (von Willebrand factor type A) domain required for RPN10 docking to the proteasome and a C-terminal half with ubiquitin-interacting motifs (UIM) involved in binding to ubiquitin chains that are attached to lysine residues of proteins directed to the proteasome for degradation (FIG. 1D). SAP05 interacted with the vWA domain but not the UIM domain of A. thaliana RPN10 (FIG. 1E). GATA18 and GATA19 proteins in which all lysines were replaced by arginines were also degraded in an SAP05-dependent manner (FIG. 1G). Moreover, SPL or GATA zinc-finger domains alone that are sufficient for SAP05 binding were degraded in the presence of SAP05 (FIG. 1H-J). These results indicate that SAP05 directly targets proteins that it interacts with for degradation and that lysine ubiquitination is not required for this degradation.

### EXAMPLE II SAP05 DEGRADES GATAS AND SPLS IN PLANTA

To investigate whether SAP05 degrades GATAs and SPLs in whole plants, we made use of existing A. thaliana lines that ectopically express GATA and SPL protein-coding regions, leading to overexpression (OE) as compared to wild-type plants. These included: (i) the full-length coding region of a Cucumis sativus L. GATA18 homologue (CsHAN1) under the control of the 35S promoter in the A. thaliana han mutant (han-2) background (ii) the full-length coding region of A. thaliana SPL5 under the control of the 35S promoter; and (iii) miR156-resistant forms of SPL11 and SPL13 (rSPL11 and rSPI13, respectively) fused with a β-glucuronidase (GUS) under the control of their native promoter. The CsHAN1-OE produced leaves with more severe serrations than those of the original han-2 line (FIG. 1A-B and 2A-B), which is consistent with the described functions of HAN, whereas introduction of SAP05 into this line alleviated the serration phenotype (compare han-2 HAN-OE to han-2 HAN-OE×35S::SAP05 plants, (FIG. 1A-B and 2A-B). Similarly, the reduction in rosette leaf production of OE lines for SPL5, rSPL11 and rSPL13 and the early bolting phenotype of SPL5-OE plants were relieved in the presence of SAP05 (FIG. 1A, C-D and 3A, C-D). These data provide evidence that SAP05 also degrades GATAs and SPLs in whole plants.

### EXAMPLE III: A FUSION PROTEIN (SAP05-NANOBODY) MEDIATES TARGET-SPECIFIC DEGRADATION

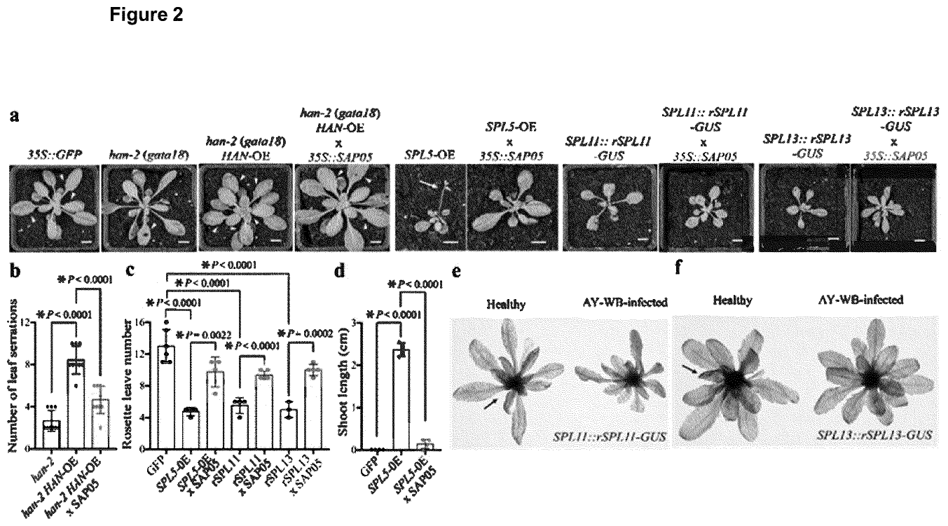

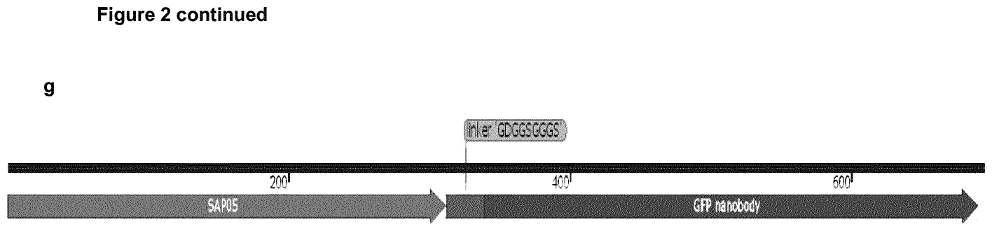

In this example, we fused SAP05 to a GFP-nanobody, a single-chain antibody domain that specifically recognizes GFP, represented in FIG. 2g. As shown in FIG. 2, this fusion protein degraded GFP in A. thaliana protoplasts (FIGS. 2 and 1K), demonstrating that it is possible to fuse a target binding moiety to SAP05 to cause selective degradation of the target protein.

### EXAMPLE IV: 26S PROTEASOME INHIBITORS MG132 AND BORTEZOMIB SUPPRESS SAP05-MEDIATED PROTEIN DESTABILIZATION

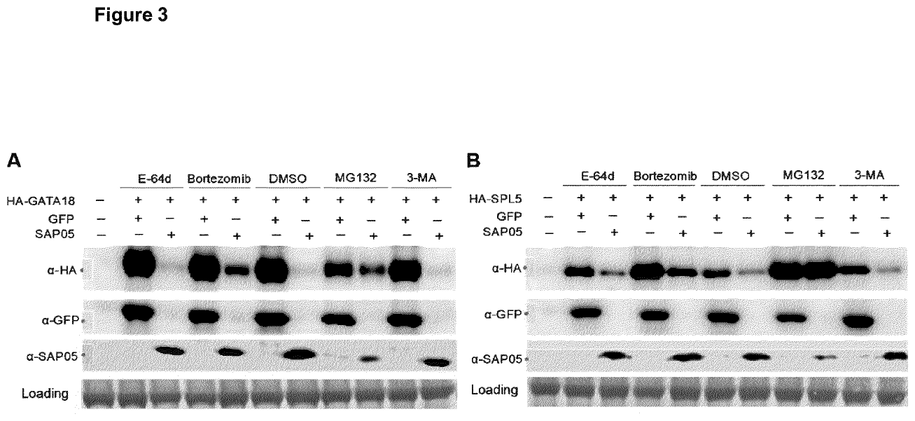

In this example, we found that the SAP05-mediated target degradation can be suppressed by the presence of 26S proteasome inhibitor MG132 and Bortezomib (FIG. 3). In contrast, the presence of autophagy inhibitors 3-MA and E-64d does not interfere with the SAP05-mediated degradation. This result suggests that the SAP05-mediated target degradation relies on the 26S proteasome but not the autophagy pathway.

### EXAMPLE V:—SAP05, ZNF DOMAIN OF SPL5 AND THE VWA DOMAIN OF ATRPN10 FORM A TERNARY COMPLEX IN VITRO

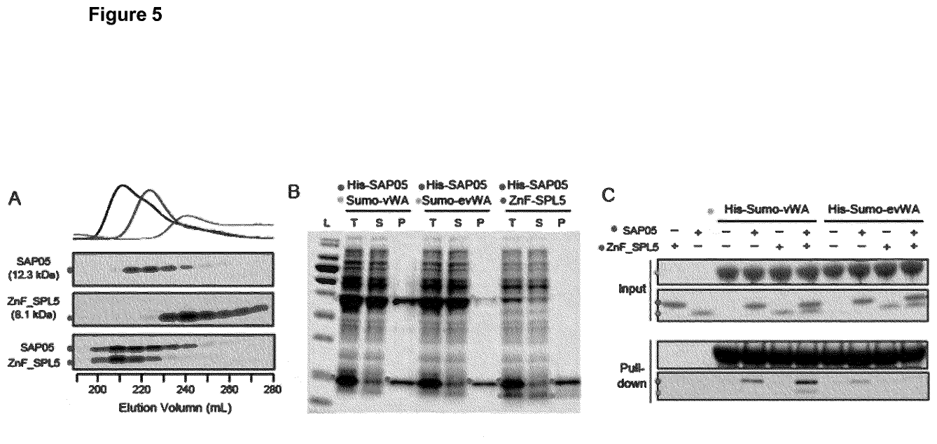

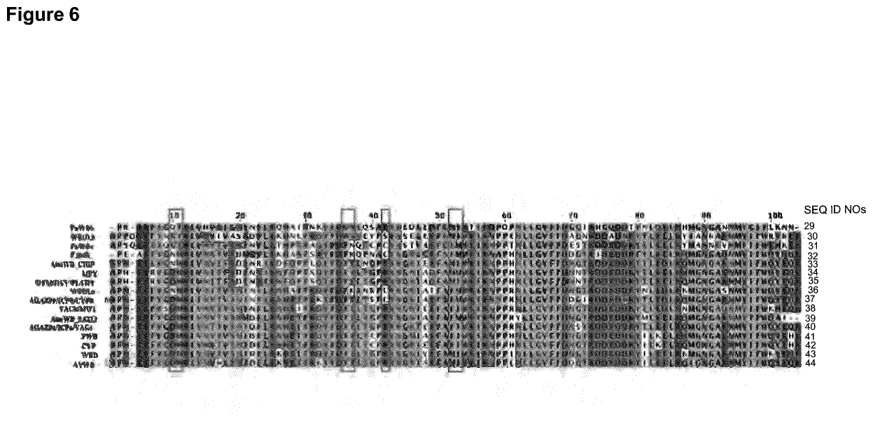

To test whether SAP05 can directly bind to the transcription factors and AtRPN10, recombinant proteins or domains were expressed and purified from E. coli to determine direct protein-protein interaction. Firstly, the interaction of SAP05 and the ZnF domain of AtSPL5 was confirmed in gel filtration chromatography (FIG. 5A). Secondly, both the vWA domain of AtRPN10 and the ZnF domain of AtSPL5 could be co-purified with His-tagged SAP05 by immobilised metal affinity chromatography (FIG. 5B), suggesting that SAP05 can bind to these two domains. Thirdly, in a His-tag pull-down assay, vWA domain was shown to pull-down SAP05 but not the ZnF domain of AtSPL5. Only when SAP05 is present, the later can be pulled down by vWA domain (FIG. 5C), suggesting that SAP05 mediates the ternary complex formation. Meanwhile, a vWA domain with a two-amino-acid substitution (evWA, 38GA39->HS) which does not interact with SAP05 in yeast two-hybrid assays also showed reduced interaction with SAP05 (FIG. 5B and 5C). These results support the idea that SAP05 mediates the ternary complex formation by direct protein-protein interaction.

These results also show that the GA residues of the vWA domain are necessary for SAP05 binding to vWA. This is also further shown in FIG. 8 (A) to (C). Comparison of multiple vWA domains of plant and animal RPN10 homologues revealed differences between the two groups in two regions corresponding to amino acids 38-39 (GA vs. HS) and 56-58 (GKG vs. K—) in the A. thaliana vWA domain (FIG. 8A). Altering these residues within A. thaliana RPN10 to those present in the M. quadrilineatus homologue, to create RPN10_38GA39->HS (m1) and RPN10_56GKG58->K (m2), resulted in a loss of SAP05 binding in Y2H assays (FIG. 8B). The RPN10 variants interacted with the A. thaliana RADIATION SENSITIVE23 (RAD23B) protein, a ubiquitin shuttle factor that binds RPN10 UIM domains (Farmer et al., 2010), indicating that the RPN10 variants are functional in the Y2H assays. In addition, SAP05 degradation assays of AtGATA18 and AtSPL5 in A. thaliana rpn10-2 protoplasts showed that these SAP05-targets were less degraded in the presence of AtRPN10 m1 compared to AtRPN10 or AtRPN10 m2 (FIG. 8C), indicating that the AtRPN10 vWA domain, and particularly the 38GA39 residues that are unique to plant versus animal RPN10 proteins, are involved in SAP05 binding and SAP05-mediated degradation of AtGATA18 and AtSPL5. For this reason, a

### EXAMPLE VI: SAP05 CAN MEDIATE PROTEIN DEGRADATION IN HUMAN 26S PROTEASOME

As shown in FIG. 7, SAP05 can mediate the degradation of proteins through the 26S proteasome. As shown in (A) co-expression of the 26S proteasome, SPL5-His, SAP05 and a plant vWA domain led to degradation of SPL5 after only 4 hours. Addition of the proteasome inhibitor, MGM132, in (B) prevented degradation of SPL5 demonstrating that SAP05 mediates protein degradation through the proteasome pathway. Figure (C) shows that SPL5 cannot be degraded in the absence of SAP05 (C) and in the absence of plant vWA (D). This result is in agreement with our finding that SAP05-mediated degradation of His-SPL5 by the human 26S proteasome occurred only in the presence of the AtRPN10 vWA domain (FIG. 7), because SAP05 is unlikely to interact with PSMD4, the human RPN10 homolog, due to the 38HS39 residues in its vWA domain. Hence, the AtRPN10 vWA domain is required for SAP05 activity. In summary, this data shows that SAP05 interacts with the human proteasome via vWA to degrade proteins.

### EXAMPLE VII: MATERIALS AND METHODS

### Yeast Two-Hybrid Analysis

The initial Y2H screen of SAP05 against the A. thaliana seedling library was performed by Hybrigenics Services SAS (Paris, France). The coding sequence of SAP05 without the secretory signal peptide was cloned into a pB27 bait plasmid as a C-terminal fusion to the LexA domain). The prey library was constructed from an A. thaliana seedling cDNA library, with pP6 as the prey plasmid. In a second yeast two-hybrid screen, the same SAP05 sequence was cloned into the pDEST32 plasmid and screened against an A. thaliana transcription factor library (pDEST22-TF). The identified interactions were further confirmed using the Matchmaker Gold yeast two-hybrid system (Clontech) or the DUALhybrid system (Dualsystems Biotech).

### Protoplast Degradation Assays

A. thaliana (Col-0) mesophyll protoplast isolation and transformation were carried out according to the methods of Yoo et al. Briefly, mesophyll protoplasts were isolated from leaves of 4-5-week-old A. thaliana plants grown under short-day conditions. For transfection, 300 μl of fresh protoplast solution (120,000 protoplasts) was transformed with 24 μg of high-quality plasmids (12 μg each for co-transfection) using the PEG-calcium method. Transfected protoplasts were incubated at room temperature (22-25° C.) for 16 h in the dark before harvest. For MG132 treatment, a final concentration of 20 μM was used during the 16-h incubation period. For detection of proteins on western blots, whole protein extracts from protoplasts were separated on NuPAGE 4-12% Bis-Tris Gels (Invitrogen) and transferred to 0.45-μm PVDF membranes (Thermo Scientific) using the Bio-Rad mini-PROTEAN Electrophoresis system. Membranes were blocked by incubation in 5% (w/v) milk power in phosphate-buffered saline and 0.1% (v/v) Tween-20 for 2 h at room temperature. Primary antibody incubation was carried out at 4° C. overnight. Antibody to SAP05 from AY-WB phytoplasma were raised to the mature part of the SAP05 protein (residues 33-135), which was produced with a 6XHis-tag into Escherichia coli and purified. The purified protein was used for raising polyclonal antibodies in rabbits (Genscript). Optimal detection of SAP05 in phytoplasma-infected plants occured at a 1:2,000 dilution of the antibody, and this dilution was used in all western blot experiments for detection of SAP05. The OptimAb HA.11 monoclonal antibody (Eurogentec) was used to detect hemagglutinin (HA)-fusion proteins at the concentration of 0.5 μg/ml. Rabbit polyclonal anti-GFP antibody (Santa Cruz Biotechnology) was used at a 1:10,000 dilution. Protein loading was visualised using Amido black staining solution (Sigma).

### Degradation Assay in Human 26S Proteasomes

For in vitro 26S proteasome degradation assay, highly purified human 26S proteasome preparation (BostonBiochem) was used immediately after thawing. 2.5 μg His-SPL5 and 5 μg SAP05 or 10 μg SAP05-vWA complex were added to 2 μg of 26S proteasome in 200 μl reaction buffer (50 mM Tris-HCl (ph 7.5), 50 mM Nacl, 10 mM MgCl2, 10% glycerol, 2 mM DTT, 5 mM ATP) and incubated at 28° C. As a control, 50 μM MG132 was added to inactivate the 26S proteasome activity. 20 μl aliquots from each reaction were collected at indicated times. Collected samples were added with SDS-PAGE loading buffer, boiled immediately and stored at −20° C. until used for western blot analysis.

### Statistical Analysis

Statistical analysis was performed in Prism 7. χ² test was used to analyse choice fecundity assay data. One-way ANOVA was used to analyse no-choice fecundity assay data with more than 2 two experimental groups, and two-tailed unpaired Student's t-test was used for other data analysis.

### REFERENCES

(1) De Smidt P C, Doan T L, Sd F, TJv B. Association of antisense oligonucleotides with lipoproteins prolongs the plasma half-life and modifies the tissue distribution. Nucleic Acids Res. 1991;19(17):4695-4700. doi: 10.1093/nar/19.17.4695
(2) Jackson A L, Bartz S R, Schelter J, Kobayashi S V, Burchard J, Mao M, et al. Expression profiling reveals off-target gene regulation by RNAi. Nat Biotechnol. 2003;21(6):635-637. doi: 10.1038/nbt831
(3) Yin H, Kauffman K J, Anderson D G. Delivery technologies for genome editing. Nat Rev Drug Discov. 2017;16(6):387. doi: 10.1038/nrd.2016.280
(4) Stein C A, Castanotto D. FDA-approved oligonucleotide therapies in 2017. Mol Ther. 2017;25(5):1069-1075. doi: 10.1016/j.ymthe.2017.03.023
(5) Mansoori B, Mohammadi A, Davudian S, Shirjang S, Baradaran B. The Different Mechanisms of Cancer Drug Resistance: A Brief Review. Adv Pharm Bull. 2017;7(3):339-348. doi:10.15171/apb.2017.041
(6) Callis J. The ubiquitination machinery of the ubiquitin system. Arabidopsis Book. 2014;12:e0174. Published 2014 Oct 6. doi:10.1199/tab.0174

1. A fusion protein comprising a von Willebrand factor type A (vWA) targeting moiety and a target-binding moiety.

2. The fusion protein of claim 1, wherein the vWA targeting moiety binds at least the HS or GA residues at positions 38 and 39 of SEQ ID NO: 2, 21, 24 or 26 or corresponding positions in a homologous sequence of vWA.

3. The fusion protein of claim 1 or 2, wherein the vWA targeting moiety is a SAP05 peptide.

4. The fusion protein of claim 3, wherein the SAP05 peptide comprises an amino acid sequence as defined in SEQ ID NO: 1 or a functional variant thereof.

5. The fusion protein of any preceding claim, wherein the fusion protein comprises at least one antibody, nanobody or antigen-binding fragment thereof or an aptamer.

6. The fusion protein of claim 5, wherein the fusion protein comprises a bispecific antibody or nanobody capable of binding vWA and a target.

7. The fusion protein of claim 5, wherein the vWA targeting moiety is a first antibody and the target-binding moiety is a second antibody and wherein the f.

8. The fusion protein of claim 4, wherein the target-binding moiety is selected from an antibody, nanobody or antigen-binding fragment thereof or an aptamer.

9. The fusion protein of claim 8, wherein the protein further comprises a linker, linking the vWA targeting moiety and target-binding moiety.

10. The fusion protein of any one of claims 1 to 9, wherein the fusion protein further comprises a RPN10 protein or a vWA domain, wherein preferably the RPN10 protein comprises an amino acid sequence as defined in SEQ ID NO: 5, 24 or 26 or a functional variant thereof and wherein the vWA domain comprises an amino acid sequence as defined in SEQ ID NO: 21 or a functional variant thereof.

11. An expression vector comprising a nucleic acid encoding the fusion peptide of any one of claims 1 to 10, or a nucleic acid comprising the nucleic acid sequence as defined in SEQ ID NO: 2 and/or 22 or a functional variant thereof.

12. A cell comprising the fusion protein of any one of claims 1 to 10 or the expression vector of claim 11.

13. The cell of claim 12, wherein the cell is a eukaryotic cell.

14. A transgenic organism expressing the expression vector of claim 11 or comprising the cell of claim 12 or 13, wherein the organism is not a human.

15. The fusion protein of any one of claims 1 to 10 or the expression vector of claim 11, for use as a medicament.

16. Use of the fusion protein of any one of claims 1 to 10, the expression construct of claim 11, a vWA targeting moiety or a SAP05 protein in targeted protein degradation.

17. The use of claim 16, wherein the targeted protein degradation is ubiquitin-independent protein degradation.

18. A method of targeted protein degradation, the method comprising applying the fusion protein of any one of claims 1 to 10, the expression vector of claim 11, or a vWA peptide and/or SAP05 protein to a sample.

19. A method of controlling the level of a target protein, the method comprising applying the fusion protein of any one of claims 1 to 10, the expression vector of claim 11, or a vWA peptide and/or a SAP05 protein or to a sample.

20. The method of claim 18 or 19, wherein a fluorescently tagged antibody is applied to the sample in order to label a protein of interest prior to applying the fusion protein, wherein the target-binding moiety of the fusion protein is specific for the fluorescent tag, and wherein degradation of the labelled protein of interest in the sample can be detected by a decrease in fluorescent signal.

21. The method of any one of claims 18 to 20, wherein the target protein is a cytosolic protein or a cell surface protein.

22. A method of gene editing, comprising:
a) introducing a CRISPR-Cas system comprising a CRISPR enzyme to a cell or organism,
b) allowing the CRISPR-Cas system to edit a gene,
c) delivering a fusion protein according to claims 1 to 10 comprising a target moiety that is specific for said CRISPR enzyme, and
d) allowing the fusion protein to degrade the CRISPR enzyme so as to inhibit any further activity of the CRISPR-Cas system.

23. The method of any one of claims 19 to 22, wherein the target protein can cause pathology in a target organism or wherein the target protein is a drug target.

24. A method of modulating a physiological response in an organism, the method comprising administering a fusion protein of any one of claims 1 to 10, an expression vector claim 11, or a vWA peptide and/or a SAP05 protein.

25. The method of claim 24, wherein the physiological response is selected from a stress response, an immune response, a hormone response or a light response.

26. A method of treating a condition in a patient in need thereof, the method comprising administering a fusion protein of any one of claims 1 to 10 or an expression vector of claim 11, or a vWA peptide and/or a SAP05 protein.

27. The method of claim 24, wherein the condition is characterised by increased expression or activity of a target protein, and the target-binding moiety is specific for said target protein.

28. A method of treating an infection caused by a microorganism, the method comprising administering a fusion protein of any one of claims 1 to 10 or an expression vector of claim 11, or a vWA peptide and/or a SAP05 protein, wherein the target binding moiety is specific for a protein expressed by a microorganism.

29. A method of increasing the immunogenicity of a protein, the method comprising administering a fusion protein of any one of claims 1 to 10 or an expression vector of claim 11, or a vWA peptide and/or a SAP05 protein, wherein the target binding moiety is specific for the protein, and wherein proteasome degradation of the protein results in increased antigen presentation of peptides degraded from the protein.

30. The method of claim 29, wherein the fusion protein is administered to a subject suffering from an infection or cancer.

31. A method for creating a protein knockout model, the method comprising administering a fusion protein of any one of claims 1 to 10 or an expression vector of claim 11, or a vWA peptide and/or a SAP05 protein to a cell or an organism.

32. The method of claim 31, where the model is a disease model where the disease is caused or characterised by a dysfunction or absence of a target protein, and the target moiety is specific for the target protein.

33. A method of identifying a degradation effect of a target protein in a biological system, the method comprising applying the fusion protein of any of claims 1 to 10, an expression vector of claim 11, or a vWA peptide and/or a SAP05 protein, to the biological system.

34. The method of claim 33, wherein the target protein is a regulatory protein, and the method further comprises performing RNA sequencing.

35. A method of increasing yield in a plant, the method comprising introducing the fusion protein of any of claims 1 to 10 or an expression vector of claim 11, or a vWA peptide and/or a SAP05 protein to the plant, wherein the target-binding moiety is specific for a target protein the expression or activity of which is negatively correlated with yield.

36. A method of reducing or removing glutens in a plant, the method comprising introducing the fusion protein of any of claims 1 to 10 or an expression vector of claim 11, or a vWA peptide and/or a SAP05 protein to the plant, wherein the target-binding moiety is specific for gliadin or glutenin.

37. A pharmaceutical composition comprising the fusion protein of any of claims 1 to 10, or an expression vector of claim 11, or a vWA peptide and/or a SAP05 protein and a pharmaceutically acceptable diluent, carrier or excipient.

38. The pharmaceutical composition of claim 37 for use in treatment of cancer, infection, a neurodegenerative disorder or a proteopathy.

39. A screening library comprising a plurality of fusion proteins according to any one of claims 1 to 10, wherein each fusion protein comprises a different target-binding moiety.

40. A method for screening target-binding moieties using the screening library of claim 39.

41. A kit comprising a fluorescently tagged antibody and a fusion protein of any one of claims 1 to 10, wherein the target-binding moiety of the fusion protein is specific for the fluorescent tag.


SEQUENCE LISTING (53 sequences)

SEQ ID NO: 1 - protein, 135 aa, Phytoplasma
MFKIKNNLLKSKIFVFILLGLFVIINNHQAMAAPNEEFVGDMRIVNVNLSNIDILKKHETFKKYFDFTLTGPRYNGNIAEFAMIWKIKNPPLNLLGVFFDDGTRDDEDDKYILEELKQIGNGAKNMYIFWQYEQK

SEQ ID NO: 2 - DNA, 408 bp, Phytoplasma
atgtttaaaatcaaaaataatttattaaaatcaaaaatatttgtatttattttattaggattatttgtaattatcaataatcatcaagcaatggctgcccctaatgaagagtttgttggcgacatgagaatagttaatgtaaatttatcaaatattgatattcttaaaaaacatgaaacatttaaaaaatattttgattttacactaactggtcctcgttataatggaaacatagcagaatttgcaatgatatggaaaattaaaaatccgcctcttaatttattaggtgttttttttgatgatggcaccagagatgatgaagatgataaatatattttagaagaattaaaacaaataggcaatggagccaaaaatatgtatattttttggcaatatgaacaaaaataa

SEQ ID NO: 3 - protein, 230 aa, Artificial Sequence
  Synthetic - SAP05 - GFP nanobody
MAPNEEFVGDMRIVNVNLSNIDILKKHETFKKYFDFTLTGPRYNGNIAEFAMIWKIKNPPLNLLGVFFDDGTRDDEDDKYILEELKQIGNGAKNMYIFWQYEQKGDGGSGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 4 - DNA, 693 bp, Artificial Sequence
  Synthetic - SAP05 - GFP nanobody
atggccccgaatgaagagtttgtgggagacatgcgaatagtaaatgtcaatctaagtaatatagatatactaaagaagcatgagacgtttaagaagtacttcgattttacactcacgggccccaggtacaatggcaatatagccgaatttgcgatgatctggaaaatcaaaaacccaccactcaacctccttggggtcttttttgatgatggtacgcgagacgacgaagatgacaaatacattctagaggagttgaaacagatcggcaacggtgcaaagaacatgtatatattctggcaatatgagcaaaagggcgatggagggagtggcggagggagcatggatcaagtacagctagtcgaatctgggggtgctctcgtgcagccaggtgggtcattgcgtttgtcttgcgctgcctcagggtttccagtcaatcgatatagcatgcgatggtacagacaagctccgggtaaggaacgagaatgggtagccggaatgagtagcgcaggtgatcgatcatcatacgaggacagcgttaagggacgtttcaccatttccagagacgatgcccgtaacacggtctatctacagatgaactctctcaagccagaggatacagccgtatattattgtaacgttaatgtcgggtttgaatactggggccagggtacgcaggtaacggtctctagttaa

SEQ ID NO: 5 - protein, 386 aa, Arabidopsis thaliana
MVLEATMICIDNSEWMRNGDYSPSRLQAQTEAVNLLCGAKTQSNPENTVGILTMAGKGVRVLTTPTSDLGKILACMHGLDVGGEINLTAAIQIAQLALKHRQNKNQRQRIIVFAGSPIKYEKKALEIVGKRLKKNSVSLDIVNFGEDDDEEKPQKLEALLTAVNNNDGSHIVHVPSGANALSDVLLSTPVFTGDEGASGYVSAAAAAAAAGGDFDFGVDPNIDPELALALRVSMEEERARQEAAAKKAADEAGQKDKDGDTASASQETVARTTDKNAEPMDEDSALLDQAIAMSVGDVNMSEAADEDQDLALALQMSMSGEESSEATGAGNNLLGNQAFISSVLSSLPGVDPNDPAVKELLASLPDESKRTEEEESSSKKGEDEKK

SEQ ID NO: 6 - DNA, 1161 bp, Arabidopsis thaliana
atggttctcgaggcgactatgatatgtatcgacaactccgagtggatgcgaaacggagattactctccgtctaggttacaggcgcaaacggaagctgttaatcttctttgcggagccaaaacccagtcgaatccggagaatacggtggggattttgacaatggctggcaaaggagttagagtattgactactcctacctctgatcttggcaaaattctggcctgtatgcacggccttgatgtgggaggagagatcaacttaaccgcagctatccagatcgcccagctagctcttaagcatcgccaaaacaagaatcaacgccaaaggattattgtttttgctggaagtccaatcaagtacgagaagaaggccctagagatagttggaaaaaggctgaagaagaatagtgtctctcttgatattgtcaatttcggggaggatgatgatgaggaaaagcctcagaaactcgaggcgctccttacagctgtgaataacaatgacggtagccacattgttcatgttccttctggagccaatgctctctcagatgtgcttctcagcacacctgtattcacgggtgatgagggtgcaagtggctatgtttctgcggcagctgctgcagcggccgcaggtggggacttcgactttggtgtggacccaaatatcgatccagaacttgctcttgcccttcgggtctccatggaggaggagagagcaagacaagaagctgctgccaagaaggcggccgatgaggcaggtcagaaagacaaagatggggacacagcttccgcctcacaggagacagttgctaggacaactgacaagaacgctgaaccaatggatgaggacagtgcgttgctagatcaggcaattgctatgtctgttggtgatgtgaatatgtcagaagcggctgatgaggaccaggatctggctttagctctgcaaatgtcaatgagtggggaagagtcaagtgaagctacaggtgctggaaacaacctcttgggaaatcaagccttcatatcgtctgttctctcatcgcttcctggggtggatccaaatgatccggcagttaaagaactactagcgtctctgccagacgagtcaaagcgtaccgaggaggaagagagtagtagcaaaaaaggcgaggatgagaagaagtga

SEQ ID NO: 7 - protein, 135 aa, Phytoplasma; identical to SEQ ID NO: 1

SEQ ID NO: 8 - DNA, 408 bp, Phytoplasma; identical to SEQ ID NO: 2

SEQ ID NO: 9 - protein, 135 aa, Phytoplasma
MFEFKKPLQIFKIGFLILLGLFIFNSSDLIAVLPEAEFVGNMRIVNSTVTDMGVLEKHASFSEYFDFKQPNACYNGNLGEFGIMWKIKNAPHNLLGVFFDDGTRIDEDDRFTLEELKQMGNGAQNMYIFWQYQQK

SEQ ID NO: 10 - DNA, 408 bp, Phytoplasma
atgtttgaattcaaaaaaccattacaaatatttaaaattggatttttgattttattgggattatttatcttcaatagttctgatttaatagcagtccttcctgaagcagaatttgtaggtaacatgaggattgttaattcaactgtgactgacatgggtgttttagaaaaacatgcatcatttagtgaatattttgattttaagcaaccaaacgcttgttataatggcaatttaggtgaatttggtattatgtggaaaatcaaaaacgcaccgcataatttattaggagttttcttcgatgatggtactagaattgatgaagatgatagatttactttagaagaattaaaacaaatgggtaatggtgctcaaaatatgtatattttttggcagtaccaacaaaaataa

SEQ ID NO: 11 - protein, 116 aa, Phytoplasma
MKIYNPNNIIMAAPSQEEIIQGTRIVNVTVSNINVLKTHPSFAQYFDFNQTCPCYNSTVAEFCIMWKIKNPPTNLLGVFFDESTRDDEDDKYSLEELKYMANNSVNMFIFWEHKEK

SEQ ID NO: 12 - protein, 128 aa, Phytoplasma
MRSLWYLTFLFIVLNMYNYQNVVVAMPPREEFIGQTRIVHVSIGNINILKQHAIFNKYFDWSLQSARYNEDLEDFSMIWTIKDPDPNLLGVFFDGGIRHGQDDTYNLQELKHMGNGANNMYCIFLKNN

SEQ ID NO: 13 - DNA, 387 bp, Phytoplasma
atgcgaagtttatggtatttaacttttttatttatagttctaaatatgtataactatcaaaatgtagttgtagctatgccgccaagagaggaatttattggccaaactagaattgttcatgtatctattggcaatattaatattttaaaacaacatgccatatttaataaatatttcgattggagtttacaaagcgctcgttataatgaagatttagaagatttcagcatgatttggacaattaaagatccagacccaaatttattaggtgttttttttgatggcggaattagacatggccaagatgatacatataatttgcaagaattaaaacatatgggtaatggtgctaataatatgtattgtatatttctaaaaaataattaa

SEQ ID NO: 14 - protein, 137 aa, Phytoplasma
MVKIKNNLLLLLNAFFAFTLLGLFLITNNQQVMAAPNEEFVGNMRIVNITVSNINILKNHATFKQYFDFKINRPCYNGNIATFAIMWKIKNPPRNLLGVFFDNGTRDDEDDKYNLEDLKKMGNGASNMYIFWQYEQK

SEQ ID NO: 15 - DNA, 414 bp, Phytoplasma
atggttaaaataaaaaataacttactattgttattgaatgcattttttgcatttactttattaggtttatttttaattactaataatcaacaagtaatggccgcccctaatgaagaatttgttggtaatatgagaatcgttaatataactgtatcaaatattaatattcttaaaaatcatgcaacatttaaacaatactttgattttaaaataaatagaccttgttataacggaaatatagcaacttttgctattatgtggaaaattaaaaatccacctcgtaatttattaggtgttttttttgataatggcactagagatgatgaagatgataaatataacttagaagatttgaaaaaaatgggaaatggtgcttcaaatatgtatattttttggcaatatgaacaaaaataa

SEQ ID NO: 16 - protein, 130 aa, Phytoplasma
MIKFYFYLYILFIFVKISTLNNIIIAAPPQDEFINGTRIVNVIVASGDILKKHNLFKQYFDWSCEYPSYNSELEEFGMIWKIKNPPENLLGVFFDAGNRDDADNKYTLEELKYIANKAKNMYIFWRYKEK

SEQ ID NO: 17 - DNA, 393 bp, Phytoplasma
atgataaaattttatttttatttatatattttatttatatttgtgaaaatttctactttgaataatattattatagctgcaccgccacaagatgaattcattaatggaactagaattgttaatgtaattgttgctagtggtgatattttaaaaaaacataatttatttaaacaatattttgattggtcttgcgaatatcctagttataattccgaattggaagaatttggtatgatttggaaaattaaaaatccacccgaaaatttattaggagttttttttgatgcaggtaatagagatgatgcagataataagtatactttagaagagttaaaatatatagctaataaggctaaaaatatgtatattttttggagatataaagaaaaataa

SEQ ID NO: 18 - protein, 4 aa, Artificial Sequence
  Synthetic - localisation signal
  misc_feature (3)..(4): Xaa can be any naturally occurring amino acid
KKXX

SEQ ID NO: 19 - protein, 4 aa, Artificial Sequence
  Synthetic - localisation signal
KDEL

SEQ ID NO: 20 - DNA, 351 bp, Phytoplasma
atgaaaatttataatccaaataatattattatggctgcgccatcgcaagaagaaatcattcaaggaactagaattgttaatgtaactgtaagtaatattaatgttttaaaaacacatccttcatttgcgcaatactttgattttaatcaaacatgtccttgttataacagtactgtagcagaattttgtattatgtggaaaattaaaaatccacctacaaatttattaggtgttttttttgatgaaagcactagagatgatgaagatgataaatattctttagaagaattaaaatatatggctaataattctgttaatatgtttattttttgggaacataaagaaaaataa

SEQ ID NO: 21 - protein, 193 aa, Arabidopsis thaliana
MVLEATMICIDNSEWMRNGDYSPSRLQAQTEAVNLLCGAKTQSNPENTVGILTMAGKGVRVLTTPTSDLGKILACMHGLDVGGEINLTAAIQIAQLALKHRQNKNQRQRIIVFAGSPIKYEKKALEIVGKRLKKNSVSLDIVNFGEDDDEEKPQKLEALLTAVNNNDGSHIVHVPSGANALSDVLLSTPVFTG

SEQ ID NO: 22 - DNA, 579 bp, Arabidopsis thaliana
atggttctcgaggcgactatgatatgtatcgacaactccgagtggatgcgaaacggagattactctccgtctaggttacaggcgcaaacggaagctgttaatcttctttgcggagccaaaacccagtcgaatccggagaatacggtggggattttgacaatggctggcaaaggagttagagtattgactactcctacctctgatcttggcaaaattctggcctgtatgcacggccttgatgtgggaggagagatcaacttaaccgcagctatccagatcgcccagctagctcttaagcatcgccaaaacaagaatcaacgccaaaggattattgtttttgctggaagtccaatcaagtacgagaagaaggccctagagatagttggaaaaaggctgaagaagaatagtgtctctcttgatattgtcaatttcggggaggatgatgatgaggaaaagcctcagaaactcgaggcgctccttacagctgtgaataacaatgacggtagccacattgttcatgttccttctggagccaatgctctctcagatgtgcttctcagcacacctgtattcacgggt

SEQ ID NO: 23 - DNA, 1143 bp, Homo sapiens
atggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaaggtgaaagagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatctgagccagccaaggaggaggatgattacgacgtgatgcaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtga

SEQ ID NO: 24 - protein, 380 aa, Homo sapiens
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEGERDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKK

SEQ ID NO: 25 - DNA, 1134 bp, Homo sapiens
atggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatctgagccagccaaggaggaggatgattacgacgtgatgcaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtga

SEQ ID NO: 26 - protein, 377 aa, Homo sapiens
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKK

SEQ ID NO: 27 - protein, 382 aa, Macrosteles quadrilineatus
MVLESTMICVDNSDYMRNGDFLPTRLQAQQDAVNIVCHSKTRSNPENNVGLLTLAKVEVLATLTSDVGRILSKLHQVQPEGNINLLTGIRIAHLALKHRQGKNHKMRIVAFVGSPVDQEEKELVKLAKRLKKEKVNVDIVSFGEEIINTDVLTSFVNTLNGKDGGGSHLVTVPPGPHLSEALISSPVIQGEDGMGGAGLGGAGFEFGVDPNEDPELALALRVSMEEQRQRQEQEARKSNTDSKEAARPDTIKEGDNEEASLERALAMSVEGQNQPTPQRQTPDFSHMTEEEQIAFAMQMSLQDSQDPGAGKESENKKDNPPETPMEVEDYAEVMNDPEFLQSVLENLPGVDPQSEAVRQAVGSLNKDKEAKEKEKKSKDGSK

SEQ ID NO: 28 - protein, 386 aa, Arabidopsis thaliana; identical to SEQ ID NO: 5

SEQ ID NO: 29 - protein, 101 aa, Phytoplasma
PREEFIGQTRIVHVSIGNINILKQHAIFNKYFDWSLQSARYNEDLEDFSMIWTIKDPDPNLLGVFFDGGIRHGQDDTYNLQELKHMGNGANNMYCIFLKNN

SEQ ID NO: 30 - protein, 104 aa, Phytoplasma
APPQDEFINGTRIVNVIVASGDILKKHNLFKQYFDWSCEYPSYNSELEEFGMIWKIKNPPENLLGVFFDAGNRDDADNKYTLEELKYIANKAKNMYIFWRYKEK

SEQ ID NO: 31 - protein, 104 aa, Phytoplasma
APSQEEIIQGTRIVNVTVSNINVLKTHPSFAQYFDFNQTCPCYNSTVAEFCIMWKIKNPPTNLLGVFFDESTRDDEDDKYSLEELKYMANNSVNMFIFWEHKEK

SEQ ID NO: 32 - protein, 102 aa, Phytoplasma
PEAEFVGNMRIVNSTVTDMGVLEKHASFSEYFDFKQPNACYNGNLGEFGIMWKIKNAPHNLLGVFFDDGTRIDEDDRFTLEELKQMGNGAQNMYIFWQYQQK

SEQ ID NO: 33 - protein, 103 aa, Phytoplasma
SPNEELVGNMRIVNVTFSDINRIKDFQPFLQYFDFTLNGPRYNGNIEEFAMIWKIKNPPHNLLGVFFDNGTRDDEDDKYTLEDLKQMGNGAKNMYIFWQYEQK

SEQ ID NO: 34 - protein, 103 aa, Phytoplasma
APHEERVGDMRIVNITFSDINSIKNFQPFSQYFDFTLTGPRYNGNIAQFAMMWKIKNPPHNLLGVFFDNNTRDDEDDKYTLEELKQMGNGAENMYIFWQYEQK

SEQ ID NO: 35 - protein, 103 aa, Phytoplasma
APHEERVGDMRIVNITFSDINSIKNFQPFSQYFDFTLTGPRYNGNIAQFAMIWKIKNPPHNLLGVFFDNNTRDDEDDKYTLEELKQMGNGAKNMYIFWQYEQK

SEQ ID NO: 36 - protein, 103 aa, Phytoplasma
APNEEFVGNMRIVNITVSNINILKNHATFKQYFDFKINRPCYNGNIATFAIMWKIKNPPRNLLGVFFDNGTRDDEDDKYNLEDLKKMGNGASNMYIFWQYEQK

SEQ ID NO: 37 - protein, 103 aa, Phytoplasma
APNEEFVGDMRIINVTVSNINIIKNHETFKKYFDFTLTSPCYNGNIAEFSIMWKIKNSPRNLLGVFFDDGIRDDEDNKYTLEDLKQMGNGAKNMYIFWQYEQK

SEQ ID NO: 38 - protein, 103 aa, Phytoplasma
APNEEFVSDMRIVNVTVSNINILKNHEIFKQYFDFTLTGPRYNGNIEEFAIIWKIKNPPRNLLGVFFDNGTRDDEDDKYTLEDLKNMGNGAQNMYIFWKHEQK

SEQ ID NO: 39 - protein, 101 aa, Phytoplasma
APNEEFVGDMRIVNVTVSNMDILKNHETFKQYFDFTLTGPRYNGNIAEFAIMWKIKNPPRYLLGVFFDNGTRDDEDDKYMLEELKQMGNGAKNMYIFWQAI

SEQ ID NO: 40 - protein, 103 aa, Phytoplasma
APNEEFVGDMRIVNTTVSNIDILKNHETFKQYFDFTLTGPRYNGNTEEFAIIWKIKNPPRNLLGVFFDNSTRDDEDDKYTLEELKQMGNGAKNMYIFWQYEQK

SEQ ID NO: 41 - protein, 103 aa, Phytoplasma
APNEEFVGDMRIVNVTVSNIDILKNHETFKQYFDFTLTCPRYNGNIAEFAIIWKIKNPPHNLLGVFFDNGTRDDEDDKYILKELKQMGNGAKNMYIFWQYEHK

SEQ ID NO: 42 - protein, 103 aa, Phytoplasma
APNEEFVGDMRIVNVTVSNIDILKNHETFKQYFDFTLTCPRYNGNIEEFAIIWKIKNPPHNLLGVFFDNGTRDDEDDKYILKELKQMGNGAKNMYIFWQYEHK

SEQ ID NO: 43 - protein, 103 aa, Phytoplasma
APNEEFVGDMRIVNVTVSNIDILKKHETFKQYFDFTLTGPRYNGNIVEFAMIWKIKNPPINLLGVFFDNGTRDDEDDKYILEELKKMGNGAKNMYIFWKYEQK

SEQ ID NO: 44 - protein, 103 aa, Phytoplasma
APNEEFVGDMRIVNVNLSNIDILKKHETFKKYFDFTLTGPRYNGNIAEFAMIWKIKNPPLNLLGVFFDDGTRDDEDDKYILEELKQIGNGAKNMYIFWQYEQK

SEQ ID NO: 45 - protein, 70 aa, Arabidopsis thaliana
MVLEATMICIDNSEWMRNGDYSPSRLQAQTEAVNLLCGAKTQSNPENTVGILTMAGKGVRVLTTPTSDLG

SEQ ID NO: 46 - protein, 70 aa, Solanum lycopersicum
MVLEATMICIDNSEWMRNGDYSPNRFQALADAVNLICGAKTQSNPENTVGILTMAGKGVRVLVTPTSDLG

SEQ ID NO: 47 - protein, 70 aa, Oryza sativa
MVLEATMICIDNSEWMRNGDYSPNRFQAQADAVNLICGAKTQSNPENTVGVMTMAGKGVRVLVTPTSDLG

SEQ ID NO: 48 - protein, 70 aa, Zea mays
MVLEATMICIDNSEWMRNGDYAPSRFQAQADAVNLICGAKTQSNPENTVGVMTMAGKGVRVLVTPTSDLG

SEQ ID NO: 49 - protein, 69 aa, Drosophila melanogaster
MVLESTMICFDNSDFQRNGDYFPTRLIVQRDGINLVCLTKLRSNPENNVGLMTLSNTVEVLATLTSDAG

SEQ ID NO: 50 - protein, 69 aa, Homo sapiens
MVLESTMVCVDNSEYMRNGDFLPTRLQAQQDAVNIVCHSKTRSNPENNVGLITLANDCEVLTTLTPDTG

SEQ ID NO: 51 - protein, 68 aa, Macrosteles quadrilineatus
MVLESTMICVDNSDYMRNGDFLPTRLQAQQDAVNIVCHSKTRSNPENNVGLLTLAKVEVLATLTSDVG

SEQ ID NO: 52 - protein, 68 aa, Bemisia tabaci
MVLESTMICVDNSDFMRNGDFIPTRLQAQQDAINVVSHSKTRSNPENNVGLLTMANVKVVSSLTSDVG

SEQ ID NO: 53 - protein, 68 aa, Myzus persicae
MVLESTMICVDNSDYMRNGDFVPTRLQAQQDAVNLVCLSKTRANPENNVGLLTLANVRVLATLTADVG